SEQUENCE LISTING (4 sequences)

SEQ ID NO: 1 - DNA, 1715 bp, Avian Influenza H5N1
  CDS (1)..(1704)
ggtaccgccaccatggagaagatcgtgctgctgttcgccatcgtgtccctggtgaagagcgaccagatctgtatcggctaccacgccaacaacagcaccgagcaggtggacaccatcatggagaaaaacgtgaccgtgacccacgcccaggacatcctggagaagaagcacaacggcaagctgtgtgacctggacggcgtgaagcccctgatcctgagggactgctccgtggccggctggctgctgggcaaccccatgtgtgacgagttcatcaacgtgcccgagtggagctacatcgtggagaaggccaaccccgtgaacgacctgtgctaccccggcgacttcaacgactacgaggagctgaagcacctgctgtcccggatcaaccacttcgagaagatccagatcatccccaagagcagctggagcagccacgaggccagcctgggcgtgtccagcgcctgcccctaccagggcaagtccagcttcttccggaacgtggtgtggctgatcaagaagaacagcacctaccccaccatcaagcggagctacaacaacaccaaccaagaggacctgctggtcctgtggggcatccaccaccccaacgacgccgccgagcagaccaagctgtaccagaaccccaccacctacatctctgtgggcaccagcaccctgaaccagcggctggtgccccggatcgccacccggagcaaggtgaacggccagagcggccggatggagttcttctggaccatcctgaagcccaacgatgccatcaacttcgagagcaacggcaacttcatcgcccccgagtacgcctacaagatcgtgaagaagggcgacagcaccatcatgaagtccgagctggagtacggcaactgtaacaccaagtgccagacccccatgggcgccatcaacagcagcatgcccttccacaacatccaccccctgaccatcggcgagtgccccaagtacgtgaagagcaacaggctggtgctggccaccggcctgaggaacagcccccagatcgagaccaggggcctgttcggcgccatcgccggcttcatcgagggcggctggcagggcatggtggacggctggtacggctaccaccacagcaatgagcagggcagcggctacgccgccgacaaggagagcacccaaaaagccatcgacggcgtcaccaacaaggtgaacagcatcatcgacaagatgaacacccagttcgaggccgtgggccgggagttcaacaacctggagcggcggatcgagaacctgaacaagaagatggaggacggcttcctggatgtgtggacctacaacgccgagctgctggtgctgatggagaacgagcggaccctggacttccacgacagcaacgtgaagaacctgtacgacaaagtgaggctgcagctgagggacaacgccaaggagctgggcaacggctgcttcgagttctaccacaagtgtgacaacgagtgtatggagtctgtgaggaacggcacctacgactacccccagtacagcgaggaggcccggctgaagcgggaggagatcagcggcgtgaaactcgagtccatcggcatctaccagatcctgagcatctacagcaccgtggccagcagcctggccctggccatcatggtggccggcctgagcctgtggatgtgctccaacggcagcctgcagtgccggatctgtatctgatagggccc

SEQ ID NO: 2 - protein, 568 aa, Avian Influenza H5N1
GTATMEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQIETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 3 - DNA, 1575 bp, Avian Influenza H5N1
  CDS (1)..(1575)
ggtaccgccaccatggagaagatcgtgctgctgttcgccatcgtgtccctggtgaagagcgaccagatctgtatcggctaccacgccaacaacagcaccgagcaggtggacaccatcatggagaaaaacgtgaccgtgacccacgcccaggacatcctggagaagaagcacaacggcaagctgtgtgacctggacggcgtgaagcccctgatcctgagggactgctccgtggccggctggctgctgggcaaccccatgtgtgacgagttcatcaacgtgcccgagtggagctacatcgtggagaaggccaaccccgtgaacgacctgtgctaccccggcgacttcaacgactacgaggagctgaagcacctgctgtcccggatcaaccacttcgagaagatccagatcatccccaagagcagctggagcagccacgaggccagcctgggcgtgtccagcgcctgcccctaccagggcaagtccagcttcttccggaacgtggtgtggctgatcaagaagaacagcacctaccccaccatcaagcggagctacaacaacaccaaccaagaggacctgctggtcctgtggggcatccaccaccccaacgacgccgccgagcagaccaagctgtaccagaaccccaccacctacatctctgtgggcaccagcaccctgaaccagcggctggtgccccggatcgccacccggagcaaggtgaacggccagagcggccggatggagttcttctggaccatcctgaagcccaacgatgccatcaacttcgagagcaacggcaacttcatcgcccccgagtacgcctacaagatcgtgaagaagggcgacagcaccatcatgaagtccgagctggagtacggcaactgtaacaccaagtgccagacccccatgggcgccatcaacagcagcatgcccttccacaacatccaccccctgaccatcggcgagtgccccaagtacgtgaagagcaacaggctggtgctggccaccggcctgaggaacagcccccagatcgagaccaggggcctgttcggcgccatcgccggcttcatcgagggcggctggcagggcatggtggacggctggtacggctaccaccacagcaatgagcagggcagcggctacgccgccgacaaggagagcacccaaaaagccatcgacggcgtcaccaacaaggtgaacagcatcatcgacaagatgaacacccagttcgaggccgtgggccgggagttcaacaacctggagcggcggatcgagaacctgaacaagaagatggaggacggcttcctggatgtgtggacctacaacgccgagctgctggtgctgatggagaacgagcggaccctggacttccacgacagcaacgtgaagaacctgtacgacaaagtgaggctgcagctgagggacaacgccaaggagctgggcaacggctgcttcgagttctaccacaagtgtgacaacgagtgtatggagtctgtgaggaacggcacctacgactacccccagtacagcgaggaggcccggctgaagcgggaggagatcagcggcgtgaaactcgag

SEQ ID NO: 4 - protein, 525 aa, Avian Influenza H5N1
GTATMEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQIETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLE

(19) United States
(12) Patent Application Publication
(10) Pub. No.: US 2007/0286873 A1
(43) Pub. Date: Dec. 13, 2007

(54) Recombinant Influenza H5 Hemagluttinin Protein And Nucleic Acid Coding Therefor

(76) Inventors: John V. Williams, Nashville, TN (US); James E. Crowe JR., Nashville, TN (US)

Correspondence Address: FULBRIGHT & JAWORSKI L.L.P., 600 CONGRESS AVE., SUITE 2400, AUSTIN, TX, 78701, US

(21) Appl. No.: 11/752,742

(22) Filed: May 23, 2007

Related U.S. Application Data
(60) Provisional application No. 60/802,667, filed on May 23, 2006.

(51) Int. Cl. A61K 39/00; A61P 43/00; C07K 14/00; C12N 15/00; C12N 15/11; C12P 21/04; G01N 33/53

(52) U.S. Cl. 424/210.1; 435/320.1; 435/69.1; 435/7.1; 536/23.1

(57) ABSTRACT

The present invention provides an improved form of a nucleic acid encoding for the influenza hemagluttinin H5N1. The optimized gene sequence permits production via recombinant means, of the H5N1 HA protein for use in diagnostic assays and vaccines.

### BACKGROUND OF THE INVENTION

The present application claims benefit of priority to U.S. Provisional Application Ser. No. 60/802,667, filed May 23, 2006, the entire contents of which are hereby incorporated by reference.

1. Field of the Invention

The present invention relates generally to the fields of molecular biology, genetics and virology. More particularly, it concerns the synthesis and use of influenza hemagluttinin H5N1-encoding nucleic acid and the production, via recombinant means, of the H5N1 protein for use in diagnostic assays and vaccines.

2. Description of Related Art

Avian H5N1 influenza is an emerging pathogen in both avian and human populations. Highly pathogenic strains of H5N1 have caused numerous outbreaks in commercial poultry flocks in recent years, with major economic consequences (Webster et al., 1986; Horimoto et al., 1995; Cauthen et al., 2000). There have been over 100 cases of human disease due to H5N1 influenza, with over 50% mortality (Beigel et al., 2005). Most of the deaths have been in previously healthy young adults and children, suggesting that H5N1 possesses significantly greater virulence than usual seasonal influenza. Virtually all cases have occurred in those with close contacts to poultry, with only one well-documented case of person-to-person transmission (Beigel et al., 2005).

Viral determinants of virulence have been established in birds and mice and include a polybasic HA cleavage site and two point mutations in HA and the PB2 polymerase subunit (Hatta et al., 2001). These have not been proven to be determinants of virulence in humans, but it is likely that they are important for highly pathogenic strains. Genetic analysis and recent studies of the 1918 influenza suggest that it also was of avian origin (Taubenberger et al., 2005; Tumpey et al., 2005; Glaser et al., 2005). During the pandemic of 1918, high mortality was observed in patients of all ages, partly due to a lack of pre-existing immunity to the then novel H1 subtype (Taubenberger et al., 2001).

Usual seasonal influenza exhibits the highest attack rates in young infants and the elderly (Zambon, 2001; Neuzil et al., 2000; Stephenson and Zambon, 2002). Studies of seasonal influenza suggest that serum virus-neutralizing antibodies are the primary mediators of protection. Resistance to influenza infection correlates with several parameters but particularly with anti-HA antibodies in the serum (Couch and Kasel, 1983; Belshe et al., 2000; Couch et al., 1986; Hobson et al., 1972; Heilman and La Montagne, 1990; Clements and Murphy, 1986; Abramson, 1999; Nichol, 2001; Wareing and Tannock, 2001; el-Madhun et al., 1998). Serum antibodies to NA prevent the spread of virus, but do not protect against infection (Couch et al., 1986). Mucosal IgA has also been shown to contribute, but the importance of mucosal antibody for prevention of lower respiratory tract disease is not fully established (Clements and Murphy, 1986; Abramson, 1999; Nichol, 2001; Wareing and Tannock, 2001; el-Madhun et al., 1998). Neutralizing serum antibodies against HA are the primary immune mediator of protection from influenza infection and illness. Thus, determination of serum neutralizing titers is important in assessing both vaccine efficacy and naturally acquired immunity.

Influenza virus-specific cellular immunity also plays a role in clearance of virus. Influenza virus-specific cytotoxic T lymphocyte (CTL) responses have been shown to reduce virus titers in the lung and decrease the rate of secondary bacterial pneumonia (Mackenzie et al., 1989; McMichael et al., 1983). Studies suggest that CD8+ T cells are more important for virus clearance than protection (McMichael et al., 1986), but adoptive transfer of influenza virus-specific CD8+ T cells in mice has been associated with protection (Taylor and Askonas, 1986). The contribution of influenza virus-specific CD4+ T cells appears to be critical for B cell co-stimulation and effective antibody production and confirmed CD4+ epitopes been identified, thus both CD4+ and CD8+ cells may contribute to protection (Doherty et al., 2001; Crowe et al., 2006; Swain et al., 2004; Brown et al., 2004). In addition, the induction of CTL responses may provide broader protection against antigenically-related virus strains and CTL-mediated heterosubtypic immunity may protect against infection with influenza A virus subtypes different than the virus initially encountered (Nguyen et al., 2000; Nguyen et al., 1999). Traditionally, vaccine trials have been focused primarily on measuring serum virus-neutralizing or HAI antibodies as a correlate of protection. However, recent studies have included determination of cell-mediated immune responses to vaccines.

In vitro virus neutralization assays are highly sensitive and specific, but are time-consuming, labor-intensive and require skilled personnel. The in vitro measurement of the ability of serum to inhibit the hemagglutination of red blood cells by virus is the hemagglutinin inhibition or HAI test. This assay also requires trained laboratory personnel. More recently, in vitro neutralization tests performed with a calorimetric readout similar to ELISA (microneutralization assays) have been used that are simpler to perform, require less serum and correlate well with titers obtained by HAI (Benne et al., 1994; Okuno et al., 1990). However, these assays still require the culture of live virus, presenting a biohazard risk. Assays measuring cellular responses present similar challenges. Thus, there is a need for improved methods of detecting both humoral and cell-based immune responses against H5N1 HA molecules, as well as improved vaccine compositions containing HA.

### SUMMARY OF THE INVENTION

Thus, in accordance with the present invention, there is provided a nucleic acid sequence comprising SEQ ID NO:1 (full length) or SEQ ID NO:3 (extracellular). The nucleic acid sequence may further comprising a promoter active in eukaryotic cells, such as a constitutive, tissue specific or inducible promoter. The nucleic acid sequence may also further comprise a nucleic acid sequence encoding a vector, such as a viral vector (a retroviral vector, an adenoviral vector, an adeno-associated viral vector, a herpesviral vector, a pox-viral vector or an influenza viral vector) or a non-viral vector. The nucleic acid sequence may be embedded in a lipid vehicle, such as a nanoparticle or a uni- or multilamellar lipid vesicle. The nucleic acid may be contained with a eukaryotic or mammalian host cell, including but not limited to an insect cell.

In another embodiment, there is provided a method of expressing a protein in a cell comprising (a) providing a mammalian host cell comprising a nucleic acid sequence comprising SEQ ID NO:1 or SEQ ID NO:3, wherein the nucleic acid sequence is under the control of a promoter active in the mammalian host cell; and (b) culturing the mammalian host cell under conditions supporting protein expression. The method may further comprise isolating the protein. Isolating may comprise one or more of solubilizing the cell, chromatography, and/or treatment with enzymes that degrade non-proteinaceous molecules. Providing may comprise transforming a mammalian host cell with a vector comprising SEQ ID NO:1 or SEQ ID NO:3 and the promoter, such as a viral vector or a non-viral vector. The mammalian host cell may express the protein transiently, or the nucleic acid sequence integrates into the genome of the mammalian host cell. The method may further comprise lyophilizing the purified protein and/or may further comprise admixing the purified protein with an adjuvant.

In yet another embodiment, there is provided an isolated recombinant influenza HA oligomeric protein retaining native influenza H5 HA structure. In still yet another embodiment, there is provided a recombinant HA oligomeric protein retaining native influenza HA structure produced according to the process having the steps (a) providing a mammalian host cell comprising a nucleic acid sequence comprising SEQ ID NO:1 or SEQ ID NO:3, wherein the nucleic acid sequence is under the control of a promoter active in the mammalian host cell; and (b) culturing the mammalian host cell under conditions supporting protein expression. In a further embodiment, there is provided a vaccine comprising a recombinant influenza HA oligomeric protein retaining native influenza HA structure and an adjuvant in a pharmaceutically acceptable buffer. In an even further embodiment, there is provided a vaccine comprising an expression construct comprising a nucleic acid sequence comprising SEQ ID NO:1 or SEQ ID NO:3 in a pharmaceutically acceptable buffer, such as a viral expression vector.

Another embodiment of the present invention is a method for inducing an immune response in a subject comprising contacting the subject a recombinant influenza HA oligomeric protein or fragment thereof retaining native influenza H5 HA structure. The contacting may comprise administering the recombinant influenza HA oligomeric protein or fragment thereof to the subject, or administering a vector encoding the recombinant influenza HA oligomeric protein or fragment thereof to the subject. The subject may be a human. The method may further comprise measuring a humoral and/or cellular immune response is the subject after contacting.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. (FIG. 1A) Schematic of H5 Influenza HA protein. (FIG. 1B) HA ectodomain construct HAΔTM. HP=heptad repeat.

FIG. 2. Analysis H5 HAΔTM by gel electrophoresis and protein sharing staining (lanes 1-3) and immunoblotting (lanes 4-10). Lane 1, mock-transfected 293F supernatant. Lane 2 and 3, successive elution fractions of column-purified H5HAΔTM. Lane 3 shows predicted monomeric, dimeric and trimeric HAΔTM. Lane 4, HAΔTM immunoblotted with anti-His MAb shows predicted monomeric, dimeric and trimeric forms. Lane 5 hMPV FΔTM. Lanes 6 and 7, successive eluted fractions of H5 HAΔTM immunoblotted with anti-H5 MAb shows similar monomeric, dimeric and trimeric forms with possible higher order multimers. Lane 8, H5 HAΔTM immunoblotted with anti-His MAb shows predicted multimeric form. Lane 9, hMPV FΔTM protein. Lane 10, H5 HAΔTM immunoblotted with anti-H5 MAb shows predicted multimeric form. Molecular weight markers in kilodaltons shown to left of each image.

FIG. 3. Denaturing, non-reducing gel immunoblotted with serum from 88-year old subject vaccinated with 2 doses of inactivated H5N1 vaccine. Lanes 1 and 3, mock-transfected 293 cell supernatant. Lanes 2 and 4, H5 HAΔTM, showing predicted monomeric, dimeric and trimeric forms. Molecular weight in kilodaltons shown to left.

FIG. 4. SDS-PAGE (reducing conditions), of MPV-G expressed in 293 cells and his-purified, blotted with anti-hMPV serum. L, ladder; 1, crude lysate; 2-3, flowthrough; 4-5 Gopt-his fractions.

FIG. 5. ELISA testing human H5 post-vaccine serum vs. HAΔTM in 4-fold dilutions.

FIG. 6. Analysis of multiple cytokines by flow cytometry. Representation of detection of four cytokines/chemokines simultaneously in CD8+ T cells. Three different populations of cells are detected: 1) ±, 2) +/+, and 3) ∓. Inclusion of only one cytokine to quantitate antigen-specific T cells to influenza A/H5N1 may underestimate the cellular response to the vaccine. The examination of a panel of cytokines does not require the use of additional cells/samples.

FIG. 7. Production of IFN-γ by influenza virus-specific T cells. The number of influenza virus-specific T cells was determined by IFN-γ production using the intracellular cytokine staining assay. Cells were stimulated for 18 hours in the presence of allantoic fluid (AF) or A/New Caledonia/20/99 (H1N1) (Flu AF). The data are expressed as the percent CD4+ or CD8+ T cells secreting IFN-γ.

FIG. 8. Immunoblots of hMPV-infected (MPV inf) or DNAFopt-transfected cell lysates. Left, denaturing, nonreducing SDS-PAGE, blotted with anti-hMPV serum. In hMPV-infected cells, F appears as three bands of 58, ˜120 and ˜200 kd, representing monomeric, dimeric and trimeric forms. The bands at ˜75 kd and ˜42 kd represents hMPV G and N proteins. In F-transfected cells, a single band is seen, representing dimeric F. Right, reducing SDS-PAGE, blotted with anti-hMPV serum. F-opt-transfected cells reveal a single band representing monomeric F. Immunoblots performed under native, nondenaturing conditions show F as a trimer (data not shown).

FIG. 9. (Left) SDS-PAGE of FoptΔTM under native conditions, immunoblotted with anti-hMPV serum shows FoptΔTM at the appropriate mw for a trimer. (Right) SDS-PAGE of FoptΔTM under reducing conditions, stained with Coomassie blue. Lane 1, molecular weight marker. 2, original supernatant from FoptΔTMtransfected 293 cells. 3, Ni-NTA column-purified fraction. 4, slight amount of FoptΔTM lost in flow-through. 5, final purified FoptΔTM.

FIGS. 10A-C. (FIG. 10A) Nasal titer of hMPV. (FIG. 10B) Lung titer of hMPV. (FIG. 10C) Reciprocal serum neutralizing antibody titers. Groups are as defined in text. Comparisons between groups were made by t-test, 2-tailed, assuring unequal variance.

FIG. 11. Trypsin digestion of HAΔTM. Denaturing, non-reducing gel immunoblotted with post-H5 vaccine serum. Trypsin concentration shown in micrograms/ml. Molecular weight in kilodaltons shown to left.

FIG. 12. Peptide N-glycosidase F digestion of HA TM. Denaturing, non-reducing gel immunoblotted with post-H5 vaccine serum. PNG-F concentration shown in units. Molecular weight in kilodaltons.

FIG. 13. ELISA titers of mice pre- and post-immunization with recombinant HAΔTM as described in the text. “Pre” refers to pre-immunization sera (9 mice), “post” refers to day 14 serum collected after 1 immunization (2 mice). “Vax” refers to positive human control serum collected from a recipient of two doses of inactivated H5N1 vaccine.

FIG. 14. Analysis of H5 HAΔTM by denaturing, non-reducing gel electrophoresis and immunoblotting. Lanes 1, 3, 5, 7 and 9, mock-transfected 293F supernatant. Lanes 2, 4, 6, 8 and 10, purified H5 HA TM. Preimmune mouse sera (Pre #1 and #2) show no reactivity with H5 HAΔTM. Post-immune mouse sera (Post #1 and #2) collected on day 14 after a single immunization with H5 HAΔTM show specific detection of H5 HAΔTM in monomeric and multimeric forms. Molecular weight markers in kilodaltons shown to left.

### DESCRIPTION OF ILLUSTRATIVE EMBODIMENTS

### I. THE PRESENT INVENTION

Highly pathogenic avian influenza viruses have caused several major outbreaks in commercial poultry flocks in recent decades (Webster et al., 1986; Horimoto et al., 1995; cauthen et al., 2000). These outbreaks have spurred research into several forms of influenza vaccines. Recombinant viral-vectored vaccines encoding influenza HA have been constructed from fowlpox and vaccinia viruses (Taylor et al., 1988; Swayne et al., 2000; Karaca et al., 2005; De et al., 1988). These vaccines have shown efficacy in chickens against both low- and high-pathogenicity strains. However, safety concerns makes translation of these results to human trials difficult. Thus, traditional inactivated reassortant vaccines grown in eggs have been the mainstay of preventive strategies in commercial poultry (Tumpey et al., 2004; Halvorson, 2002).

Currently no vaccine is available to protect humans against the H5N1 virus that is being seen in Asia. However, vaccine development efforts are under way. Researchers at NIAID began studies to test a vaccine to protect humans against H5N1 virus began in April 2005. Sites in Rochester, N.Y., Baltimore and Los Angeles will enroll a total of healthy adults. The clinical sites are part of the NIAID-sponsored Vaccine and Treatment Evaluation Units (VTEU). Sanofi Pasteur (Swiftwater, Pa.) manufactured the trial vaccine, which is an inactivated vaccine made from an H5N1 virus isolated in Southeast Asia in 2004. This Phase I trial will test the vaccine's safety and ability to generate an immune response in 450 healthy adults. Researchers are also working on a vaccine against H9N2, another bird flu virus subtype.

Recent studies have reported the use of reverse-engineering to produce vaccine strains bearing modified genes to attenuate virulence (Subbarao et al., 2003; Liu et al., 2003). The reverse-engineering technique is very promising in that it allows vaccines to be “tailor-made⇄ to respond to antigenic variation in field strains, and the potential to modify virus genes to alter virulence or replication characteristics (Subbarao et al., 2003; Horimoto et al., 2003; Stech et al., 2005; Lu et al., 2005; Quinlivan et al., 2005).

However, major limitations of both traditional and reverse engineering approaches are (a) the requirement to develop vaccine seed strains that replicate to high titers in embryonated eggs; (b) the necessity for vaccine production in eggs, where one egg yields approximately one dose; and (c) the purification required for egg-produced vaccine and concerns regarding poultry-associated adventitious agents. Recent studies of cell-culture produced influenza vaccines may alleviate some of these obstacles (Oxford et al., 2005; Ghendon et al., 2005; Palker et al., 2004).

Recombinant protein subunit vaccination is a strategy that has been highly successful for hepatitis B vaccine, which is produced in yeast (Garrison and baker, 1991). Recombinant production allows strict quality control of all vaccine components and more straightforward quantitation of lot-to-lot variation. Recombinant influenza hemagglutinin has been produced using baculovirus constructs infecting insect cells (Wang et al., 2005; Vanlandschoot et al., 1996; Kuroda et al., 1986). Baculovirus-expressed HA proteins have been tested in mice and chickens and were immunogenic and protective (Brett and Johansson, 2005; Abe et al., 2003; Crawford et al., 1999; Olsen et al., 1997). In subsequent human clinical trials, baculovirus-expressed HA stimulated humoral immune responses in human vaccine trials, but required high doses (Treanor et al., 2001; Powers et al., 1997; Lakey et al., 1996; Treanor et al., 1996). One trial tested baculovirus-expressed H5 HA and detected neutralizing antibody responses to a titer of 1:80 or greater in 52% of subjects after two doses of 90 μg The requirement for such high doses (45-135 μg HA) compared to inactivated influenza vaccine (15 μg HA) presents a barrier to producing sufficient vaccine for large populations in the event of a pandemic. The reason for the decreased immunogenicity of the baculovirus-expressed protein is not clear. It may be due to a lack of previous exposure to H5 subtype virus in the subjects, who therefore would have experienced a primary rather than a primed memory response. Alternative adjuvants may be more effective at inducing robust responses to neoantigens.

Another possibility is that glycosylation of the baculovirus protein may differ from the glycosylation of wild-type hemagglutinin in mammalian cells. Studies have shown that N-linked glycosylation of HA in insect cells differs in both the pattern and nature of the oligosaccharides attached to the nascent protein (Wagner et al., 1996a; Wagner et al., 1996b; Kuroda et al., 1991; Kuroda et al., 1990). Crystal structures of HA have only been determined for bromelain-cleaved forms purified from infected mammalian cells (Carr and Kim, 1993; Gamblin et al., 2004; Ha et al., 2003; a et al., 2002). Thus, it is possible that the structure of baculovirus-expressed HA differs in immunologically important ways from wild-type HA. The present inventors hypothesized that HA expressed in mammalian cells will exhibit glycosylation, folding and antigenic structure more similar to wild-type HA in viruses that infect humans, and will be more immunogenic in mammals.

Highly pathogenic avian influenza viruses represent a major threat to human health. A pandemic due to the virulent H5N1 virus currently circulating would have devastating consequences on human health, society and economy globally. Current methods of vaccine production are limited by virus culture and egg-based production, require months to yield a final product and are not adequate to generate sufficient vaccines for large populations. Furthermore, the rapid evolution of influenza viruses makes the likelihood of antigenically variant strains arising quite high, necessitating development of a new vaccine. It is worth noting that these problems exist with vaccines for seasonal influenza as well, which is a major cause of morbidity and mortality during annual winter epidemics. Recombinant protein vaccines offer solutions to many of these difficulties.

The present inventors have developed a novel strategy for generating and purifying viral glycoproteins in a mammalian cell culture-based system, and have successfully used this approach for human metapneumovirus (hMPV) and respiratory syncytial virus (RSV). Their experience with attempts to express paramyxovirus fusion proteins showed that native glycoprotein sequences do not express well in mammalian culture systems from plasmid cDNAs. Although native viral glycoproteins do not express well from plasmids in culture, or in animals following DNA immunization, the genes do express well when transcription is driven by viral polymerases (such as in natural infection or when expressed from vaccinia virus constructs). Plasmid-based transcription differs from these systems in that plasmid DNA transcription occurs in the nucleus, whereas paramyxoviruses and vaccinia virus replicate solely in the cytoplasm. This difference suggests that nuclear import of DNA or export of nascent mRNA transcripts is a limiting step in plasmid-based expression. In addition, analysis of many viral glycoprotein genes, including the H5 influenza HA gene, reveals a large number of codons rarely used in mammalian genes, and many AT-rich regions. These features contribute to mRNA instability and hinder recombinant expression in mammalian cells.

A sequence optimization procedure resynthesizes the entire open reading frame of the gene, altering suboptimal nucleotide domains while preserving the translated amino acid sequence. This technique has been used to greatly enhance both mammalian expression and immunogenicity of the HIV gag gene and RSV F gene (Li et al., 1998; Deml et al., 2001). The inventors have utilized two methods to enhance expression of the H5 influenza HA gene in mammalian cells: 1) codon optimization for expression in human cells, and 2) optimization of mRNA stability and structure for optimal transcription. The result is construct encoding H5 influenza HA (full-length and truncated) expressed at high levels in cultured mammalian cells, thereby allowing for the production of highly pure, conformationally-accurate H5 influenza HA protein. Because constructs expressed in eukaryotic cells are amenable to large-scale bioreactor fermentation production, and can easily be changed with site-directed mutagenesis to reflect mutations occurring in circulating viruses, the problems of supply, adventitious agents and purification associated with embryonated, egg-based antigen production will be lessened.

### II. INFLUENZA A VIRUSES

A. General

Influenza viruses that infect birds are called avian influenza viruses, or influenza A. Only influenza A viruses infect birds, and all known subtypes of influenza A viruses can infect birds. Avian influenza viruses circulate among birds worldwide. Certain birds, particularly water birds, act as hosts for influenza viruses by carrying the virus in their intestines and shedding it. Infected birds shed virus in saliva, nasal secretions, and feces. Susceptible birds can become infected with avian influenza virus when they have contact with contaminated nasal, respiratory, or fecal material from infected birds. Fecal-to-oral transmission is the most common mode of spread between birds.

Most often, the wild birds that are host to the virus do not get sick, but they can spread influenza to other birds. Infection with certain avian influenza A viruses (for example, some H5 and H7 strains) can cause widespread disease and death among some species of domesticated birds. Domesticated birds may become infected with avian influenza virus through direct contact with infected waterfowl or other infected poultry, or through contact with surfaces (such as dirt or cages) or materials (such as water or feed) that have been contaminated with virus. People, vehicles, and other inanimate objects such as cages can be vectors for the spread of influenza virus from one farm to another. When this happens, avian influenza outbreaks can occur among poultry.

Avian influenza outbreaks among poultry occur worldwide from time to time. Since 1997, for example, more than 16 outbreaks of H5 and H7 influenza have occurred among poultry in the United States. The U.S. Department of Agriculture monitors these outbreaks. Low pathogenic forms of avian influenza viruses are responsible for most avian influenza outbreaks in poultry. Such outbreaks usually result in either no illness or mild illness (e.g., chickens producing fewer or no eggs), or low levels of mortality. When highly pathogenic influenza H5 or H7 viruses cause outbreaks, between 90% and 100% of poultry can die from infection. Animal health officials carefully monitor avian influenza outbreaks in domestic birds for several reasons: (a) the potential for low pathogenic H5 and H7 viruses to evolve into highly pathogenic forms; (b) the potential for rapid spread and significant illness and death among poultry during outbreaks of highly pathogenic avian influenza; and (c) the economic impact and trade restrictions from a highly pathogenic avian influenza outbreak. When avian influenza outbreaks occur in poultry, quarantine and depopulation (or culling) and surveillance around affected flocks is the preferred control and eradication option.

Although influenza A viruses usually do not infect humans, but have been know to do so, as well as infecting other hosts including pigs, whales, horses, and seals. More than 200 confirmed cases of human infection with avian influenza viruses have been reported since 1997. Most cases of avian influenza infection in humans are thought to have resulted from direct contact with infected poultry or contaminated surfaces. However, there is still a lot to learn about how different subtypes and strains of avian influenza virus might affect humans. For example, it is not known how the distinction between low pathogenic and highly pathogenic strains might impact the health risk to humans.

Subtypes that have caused widespread illness in people either in the past or currently are H3N2, H2N2, H1N1, and H1N2. H1N1 and H3N2 subtypes also have caused outbreaks in pigs, and H7N7 and H3N8 viruses have caused outbreaks in horses. Influenza A viruses normally seen in one species sometimes can cross over and cause illness in another species. For example, until 1998, only H1N1 viruses circulated widely in the U.S. pig population. However, in 1998, H3N2 viruses from humans were introduced into the pig population and caused widespread disease among pigs. Most recently, H3N8 viruses from horses have crossed over and caused outbreaks in dogs. Avian influenza A viruses may be transmitted from animals to humans in two main ways: directly from birds or from avian virus-contaminated environments to people, or through an intermediate host, such as a pig.

Because of concerns about the potential for more widespread infection in the human population, public health authorities closely monitor outbreaks of human illness associated with avian influenza. To date, human infections with avian influenza A viruses detected since 1997 have not resulted in sustained human-to-human transmission. However, because influenza A viruses have the potential to change and gain the ability to spread easily between people, monitoring for human infection and person-to-person transmission is important. Confirmed instances of avian influenza viruses infecting humans since 1997 include:
H5N1, Hong Kong, Special Administrative Region, 1997: Highly pathogenic avian influenza A (H5N1) infections occurred in both poultry and humans. This was the first time an avian influenza A virus transmission directly from birds to humans had been found. During this outbreak, 18 people were hospitalized and six of them died. To control the outbreak, authorities killed about 1.5 million chickens to remove the source of the virus. Scientists determined that the virus spread primarily from birds to humans, though rare person-to-person infection was noted.
H9N2, China and Hong Kong, Special Administrative Region, 1999: Low pathogenic avian influenza A (H9N2) virus infection was confirmed in two children and resulted in uncomplicated influenza-like illness. Both patients recovered, and no additional cases were confirmed. The source is unknown, but the evidence suggested that poultry was the source of infection and the main mode of transmission was from bird to human. However, the possibility of person-to-person transmission could not be ruled out. Several additional human H9N2 infections were reported from China in 1998-99.
H7N2, Virginia, 2002: Following an outbreak of H7N2 among poultry in the Shenandoah Valley poultry production area, one person was found to have serologic evidence of infection with H7N2.
H5N1, China and Hong Kong, Special Administrative Region, 2003: Two cases of highly pathogenic avian influenza A (H5N1) infection occurred among members of a Hong Kong family that had traveled to China. One person recovered, the other died. How or where these two family members were infected was not determined. Another family member died of a respiratory illness in China, but no testing was done.
H7N7, Netherlands, 2003: The Netherlands reported outbreaks of influenza A (H7N7) in poultry on several farms. Later, infections were reported among pigs and humans. In total, 89 people were confirmed to have H7N7 influenza virus infection associated with this poultry outbreak. These cases occurred mostly among poultry workers. H7N7-associated illness included 78 cases of conjunctivitis (eye infections) only; 5 cases of conjunctivitis and influenza-like illnesses with cough, fever, and muscle aches; 2 cases of influenza-like illness only; and 4 cases that were classified as “other.” There was one death among the 89 total cases. It occurred in a veterinarian who visited one of the affected farms and developed acute respiratory distress syndrome and complications related to H7N7 infection. The majority of these cases occurred as a result of direct contact with infected poultry; however, Dutch authorities reported three possible instances of transmission from poultry workers to family members. Since then, no other instances of H7N7 infection among humans have been reported.
H9N2, Hong Kong, Special Administrative Region, 2003: Low pathogenic avian influenza A (H9N2) infection was confirmed in a child in Hong Kong. The child was hospitalized and recovered.
H7N2, New York, 2003: In November 2003, a patient with serious underlying medical conditions was admitted to a hospital in New York with respiratory symptoms. One of the initial laboratory tests identified an influenza A virus that was thought to be H1N1. The patient recovered and went home after a few weeks.

Subsequent confirmatory tests conducted in March 2004 showed that the patient had been infected with avian influenza A (H7N2) virus.
H7N3 in Canada, 2004: In February 2004, human infections of highly pathogenic avian influenza A (H7N3) among poultry workers were associated with an H7N3 outbreak among poultry. The H7N3-associated, mild illnesses consisted of eye infections.
H5N1, Thailand and Vietnam, 2004, and other outbreaks in Asia during 2004 and 2005: In January 2004, outbreaks of highly pathogenic influenza A (H5N1) in Asia were first reported by the World Health Organization. Visit the Avian Influenza section of the World Health Organization Web site for more information and updates.
   The reported symptoms of avian influenza in humans have ranged from typical influenza-like symptoms (e.g., fever, cough, sore throat, and muscle aches) to eye infections (conjunctivitis), pneumonia, acute respiratory distress, viral pneumonia, and other severe and life-threatening complications.

B. Strains, Subtypes and Genetic Diversity

There is substantial genetic differences between the subtypes that typically infect both people and birds. Within subtypes of avian influenza A viruses there also are different strains. Avian influenza A H5 and H7 viruses can be distinguished as “low pathogenic” and “high pathogenic” forms on the basis of genetic features of the virus and the severity of the illness they cause in poultry; influenza H9 virus has been identified only in a “low pathogenicity” form. Each of these three avian influenza A viruses (H5, H7, and H9) theoretically can be partnered with any one of nine neuraminidase surface proteins; thus, there are potentially nine different forms of each subtype (e.g., H5N1, H5N2, H5N3, H5N9), each further affecting pathogenicity.

Influenza A viruses have eight separate gene segments. The segmented genome allows influenza A viruses from different species to mix and create a new influenza A virus if viruses from two different species infect the same person or animal. For example, if a pig were infected with a human influenza A virus and an avian influenza A virus at the same time, the new replicating viruses could mix existing genetic information (reassortment) and produce a new virus that had most of the genes from the human virus, but a hemagglutinin and/or neuraminidase from the avian virus. The resulting new virus might then be able to infect humans and spread from person to person, but it would have surface proteins (hemagglutinin and/or neuraminidase) not previously seen in influenza viruses that infect humans. This type of major change in the influenza A viruses is known as antigenic shift. Antigenic shift results when a new influenza A subtype to which most people have little or no immune protection infects humans. If this new virus causes illness in people and can be transmitted easily from person to person, an influenza pandemic can occur. It is possible that the process of genetic reassortment could occur in a human who is co-infected with avian influenza A virus and a human strain of influenza A virus. The genetic information in these viruses could reassort to create a new virus with a hemagglutinin from the avian virus and other genes from the human virus. Theoretically, influenza A viruses with a hemagglutinin against which humans have little or no immunity that have reasserted with a human influenza virus are more likely to result in sustained human-to-human transmission and pandemic influenza. Therefore, careful evaluation of influenza viruses recovered from humans who are infected with avian influenza is very important to identify reassortment if it occurs.

C. Detection

Conventional diagnostic tools, cell culture, and serologic testing for influenza viruses require from 2 days to 2 weeks for results; thus, they are less useful in making therapeutic and infection control decisions. On the other hand, commercially available rapid antigen tests such as Directigen Flu A+B (Becton Dickinson, Sparks, N.J., USA) or Binax NOW (Binax Inc., Portland, Me., USA) are rapid and simple, but subtyping of viruses is not feasible. Molecular diagnosis of influenza by reverse transcription-polymerase chain reaction (RT-PCR) provides a sensitive and rapid means for detection and has facilitated the typing and subtyping of viruses. Previous researchers developed tests to detect H5N1 virus by using conventional RT-PCR and confirmed the results by Southern blot analysis or restriction fragment length polymorphism-based strategy. A real-time RT-PCR based on the avian H5 gene was developed, but not evaluated on human clinical specimens.

Enders et al. (2000) developed a sensitive and rapid real-time reverse transcription-polymerase chain reaction (RT-PCR) assay to detect influenza A H5N1 virus in clinical samples. This assay was evaluated with samples from H5N1-infected patients and demonstrated greater sensitivity and faster turnaround time than nested RT-PCR. When evaluated using clinical samples from patients infected with H5N1 in Hong Kong and Vietnam, they found it was more sensitive and faster in detecting the virus than the nested RT-PCR that we used previously.

This real-time RT-PCR is a multiplex assay that employs a mixture of 2 sets of inhouse designed primers and dual-labeled fluorescent probes that specifically target 2 different regions of the HA gene of H5N1. To test for cross-reactivity, RNA was extracted from isolates or persons with human influenza A H1, H3, H9 subtypes; influenza B; human CoV 229E and OC43; respiratory syncytial virus; rhinoviruses; and enteroviruses. The RNA was then tested by real-time RT-PCR. The results showed that the assay was specific for the H5 subtype. The detection sensitivity of the real-time assay was compared with that of 2 other assays by analyzing a serial 10-fold dilution of viral stock with 10⁸ tissue culture infective dose (TCID)₅₀/mL of rhabdomyosarcoma cell-culture fluid of a recent H5N1 isolate (A/Viet Nam/1194/2004). The results showed that this assay was the most sensitive of the 3 assays, 10-fold more sensitive than nested RT-PCR.

### III. PROTEINS AND PURIFICATION

The H5N1 HA protein that is produced by the nucleic acids of the present invention will contain a primary sequence (SEQ ID NO:2 and 4) that is identical to that found in natural strains. Using the same codon preferences and mRNA stability rules, it also is possible design related proteins that have amino acid substitutions, similar to those occurring in nature (see discussion of antigenic drift above), thereby permitting vaccines and detection methods to adapt to the changing viral threat. Recombinant production of these proteins, as well as their purification and formulation into vaccines, is discussed elsewhere in this document.

A. Fragments and Peptides of H5N1 HA Proteins

In addition to the use of full length H5N1 HA sequences, the present invention contemplates the use of various fragments and truncated versions of H5N1 HA proteins, including a soluble version that lacks the transmembrane domain of the native protein. For example, a portion of the protein as set forth in SEQ ID NO:2 or SEQ ID NO: 4 may be used in various embodiments of the invention. In certain embodiments, a fragment of the may comprise, but is not limited to about 50, about 75, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 450, about 500, or about 600 residues, and any range derivable therein.

It also will be understood that such partial sequences, along with full length H5N1 HA proteins, may be joined or fused to additional residues, such as additional N— or C-terminal amino acids, and yet still be essentially as set forth in one of the sequences disclosed herein. One example is a carrier protein that can improve immunogenicity of the viral sequences.

B. In vitro Production of H5N1 HA Polypeptides or Fragments

Various types of expression vectors are known in the art that can be used for the production of protein products (discussed further below). Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth). Non-anchorage dependent or suspension cultures from continuous established cell lines are the most widely used means of large-scale production of cells and cell products. However, suspension cultured cells have limitations, such as tumorigenic potential and lower protein production than adherent cells. In further aspects of the invention, other protein production methods known in the art may be used, including but not limited to prokaryotic, yeast, and other eukaryotic hosts such as insect cells and the like.

C. Protein Purification

It may be desirable to purify H5N1 HA polypeptides or fragments thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, hydrophobic interaction chromatography, exclusion chromatography; polyacrylamide gel electrophoresis; isoelectric focusing. A particularly efficient method of purifying peptides is fast protein liquid chromatography (FPLC).

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, about 96%, about 97%, about 98%, about 99% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme.

### IV. NUCLEIC ACIDS AND EXPRESSION CONSTRUCTS

The term “gene” is used here to refer to the nucleic acid giving rise to a functional protein, polypeptide, or peptide-encoding unit, in this case an H5N1 HA protein. In addition to the full length gene, which may contain non-coding sequences, the polynucleotides of the present invention contemplate shorter lengths that comprise less than all of a complete HCV polypeptide, including about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 600, 650, 700, 750, 800, 900, 1000, 1100, 1200, 1300, 1400, 1500 or more nucleotides, nucleosides, or base pairs. Such sequences may be identical or complementary to all or part of SEQ ID NO:1 and SEQ ID NO:3.

In particular embodiments, the invention concerns isolated nucleic acid segments and recombinant vectors incorporating DNA sequences that encode HCV envelope polypeptides or peptides. Such vectors used in the present invention, regardless of the length of the coding sequence itself, may be combined with other DNA or RNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

A. Vectors Encoding H5N1 HA Proteins

The present invention encompasses the use of vectors that encode all or part of one or more H5N1 HA polypeptides. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). In particular embodiments, gene therapy or immunization vectors are contemplated. One of skill in the art would be well equipped to construct a vector through standard recombinant techniques, which are described in Maniatis et al. (1990) and Ausubel et al. (1996), both incorporated herein by reference.

The term “expression vector” or “expression construct” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

B. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” means that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the nucleic acid segment in the cell type, organelle, and organism chosen for expression. Those of skill in the art of molecular biology generally know the use of promoters, enhancers, and cell type combinations for protein expression, for example, see Sambrook et al. (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or exogenous, i.e., from a different source than the H5N1 HA sequence. In some examples, a prokaryotic promoter is employed for use with in vitro transcription of a desired sequence. Prokaryotic promoters for use with many commercially available systems include T7, T3, and Sp6.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki et al., 1998), D1A dopamine receptor gene (Lee et al., 1997), insulin-like growth factor II (Wu et al., 1997), human platelet endothelial cell adhesion molecule-1 (Almendro et al., 1996).

C. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome-scanning model of 5′ methylated Cap dependent translation and begin translation at internal sites (Pelletier and Sonenberg, 1988). IRES elements from two members of the picomavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

D. Multiple Cloning Sites

Vectors can include a multiple cloning site (MCS), which is a nucleic acid region that contains multiple restriction enzyme sites, any of which can be used in conjunction with standard recombinant technology to digest the vector (see Carbonelli et al., 1999; Levenson et al., 1998; and Cocea, 1997; all incorporated herein by reference). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

E. Termination/Polyadenylation Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

F. Origins of Replication

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

G. Selectable and Screenable Markers

In certain embodiments of the invention, the cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

H. Host Cells

As used herein, the terms “cell,” “cell line,” and “cell culture” may be used interchangeably. All of these terms also include their progeny, which refers to any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector, expression of part or all of the vector-encoded nucleic acid sequences, or production of infectious viral particles. Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either an eukaryotic or prokaryotic host cell, particularly one that is permissive for replication or expression of the vector.

I. Expression Systems

Numerous expression systems exist that comprise at least all or part of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM from CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. The Tet-On™ and Tet-Off™ systems from CLONTECH® can be used to regulate expression in a mammalian host using tetracycline or its derivatives. The implementation of these systems is described in Gossen et al. (1992) and Gossen et al. (1995), and U.S. Pat. No. 5,650,298, all of which are incorporated by reference.

INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

J. Introduction of Nucleic Acids into Cells

In certain embodiments, a nucleic acid may be introduce into a cell in vitro for production of polypeptides or in vivo for immunization purposes. There are a number of ways in which nucleic acid molecules such as expression vectors may be introduced into cells. In certain embodiments of the invention, the expression vector comprises an HCV infectious particle or engineered vector derived from an HCV genome. In other embodiments, an expression vector known to one of skill in the art may be used to express an HCV polypeptide. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986).

“Viral expression vector” is meant to include those vectors containing sequences of that virus sufficient to (a) support packaging of the vector and (b) to express a polynucleotide that has been cloned therein. In this context, expression may require that the gene product be synthesized. A number of such viral vectors have already been thoroughly researched, including adenovirus, adeno-associated viruses, retroviruses, herpesviruses, and vaccinia viruses. A specific example is an attenuated or non-virulent influenza virus vector.

Delivery may be accomplished in vitro, as in laboratory procedures for transforming cells lines, or in vivo or ex vivo, as in the treatment of certain disease states. One mechanism for delivery is via viral infection where the expression vector is encapsidated in an infectious viral particle. Several non-viral methods for the transfer of expression vectors into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), liposome (Ghosh and Bachhawat, 1991; Kaneda et al., 1989) and receptor-mediated transfection (Wu and Wu, 1987; Wu and Wu, 1988). Some of these techniques may be successfully adapted for in vivo or ex vivo use.

In certain embodiments, e.g., in vitro transformation of cells, the polynucleotide encoding an HCV gene may be stably integrated into the genome of the cell. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression vector is delivered to a cell and where in the cell the nucleic acid remains is dependent on the type of expression vector employed.

### V. VACCINES AND IMMUNIZATION

A. Vaccine Formulations

Pharmaceutical compositions including H5N1 HA polypeptides will be formulated along the line of typical pharmaceutical drug and biological preparations. A discussion of formulations may be found in Remington's Pharmaceutical Sciences (1990). The percentage of active compound in any pharmaceutical preparation is dependent upon both the activity of the compound, in this case the ability of an H5N1 HA vaccine to stimulate an immune response against H5N1 infection. Typically, such compositions should contain at least 0.1% active compound. The percentage of the compositions and preparations may, of course, be varied and may conveniently be between about 2 to about 60% of the weight of the unit. The amount of active compounds in such therapeutically useful compositions is such that a suitable dosage will be obtained.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy injection is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, phenylmecuric nitrate, m-cresol, and the like. In many cases, it will be preferable to use isotonic solutions, for example, sugars or sodium chloride. Prolonged absorption of the injectable compositions can be brought about by the use in the compositions of agents delaying absorption, for example, aluminum monostearate, and gelatin.

Sterile injectable solutions are prepared by incorporating the active compounds in the required amount in the appropriate solvent with various of the other ingredients enumerated above, as required, followed by sterile filtration. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques that yield a powder of the active ingredient plus any additional desired ingredient from a previously sterile-filtered solution thereof.

The phrases “pharmaceutically acceptable” or “pharmacologically acceptable” refer to molecular entities and compositions that do not produce an adverse, allergic, or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Peptides or polypeptides may be administered in a dose that can vary from 0.01, 0.05, 0.1, 0.5, 1, 5, 10, 20, 30, 40, 50, 60, 70, 80, 90, 100 mg/kg of weight to 50, 60, 70, 80, 90, 100, 110, 120, 130, 140, 150, 160, 170, 180, 190, 200 mg/kg of weight in one or more daily, weekly, monthly, or yearly administrations during one or various days, weeks, months, or years. For a vaccine, the goal will be to develop a formulation that elicits protective immunity in as few doses as possible, hopefully a single dose. It is possible that booster doses will be required either for the primary immunization or for repeated immunization as the initial immune response wanes. The antigens or genes encoding antigens can be administered by parenteral injection (intravenous, intraperitoneal, intramuscular, subcutaneous, intracavity, intradermal or transdermic).

For viral vectors, particularly attenuated viral vectors, one generally will prepare a viral vector stock of high titer. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, 1×10¹², 1×10¹³ or 1×10¹⁴ infectious particles to the patient. Similar figures may be extrapolated for bacterial host delivery systems, or for liposomal or other non-viral formulations by comparing relative uptake efficiencies. Formulation as a pharmaceutically acceptable composition is discussed below.

Protein vaccines often employ an immunogenic carrier protein, such as hepatitis B surface antigen, keyhole limpet hemocyanin or bovine serum albumin. Methods for performing this conjugation are well known in the art. Other immunopotentiating compounds are also contemplated for use with the compositions of the invention such as polysaccharides, including chitosan, which is described in U.S. Pat. No. 5,980,912, hereby incorporated by reference. The vaccine may further comprise an adjuvant, such as alum, Bacillus Calmette-Guerin, agonists and modifiers of adhesion molecules, tetanus toxoid, imiquinod, montanide, MPL, and QS21.

In a particular embodiment of the invention, the HCV vaccine may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991).

Current in vivo lipid delivery methods use subcutaneous, intradermal, intratumoral, or intracranial injection to avoid the toxicity and stability problems associated with cationic lipids in the circulation. The DOTAP:cholesterol lipid formulation is said to form a unique structure termed a “sandwich liposome.” This formulation is reported to “sandwich” DNA between an invaginated bi-layer or ‘vase’ structure. Beneficial characteristics of these lipid structures include a positive ρ, colloidal stabilization by cholesterol, two-dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990).

B. Vaccination Protocols

The active compounds of the present invention can be formulated for parenteral administration, e.g., formulated for injection via the intradermal, intravenous, intramuscular, intrathoracic, sub-cutaneous, or even intraperitoneal routes. Administration by the intradermal and intramuscular routes are specifically contemplated. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intradermal, and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, Remington's Pharmaceutical Sciences, 1990). Some variation in dosage will necessarily occur depending on the age and possibly medical condition of the subject being treated. The person responsible for administration will, in any event, determine the appropriate dose for the individual subject.

In many instances, it will be desirable to have several or multiple administrations of the vaccine. The compositions of the invention may be administered 1, 2, 3, 4, 5, 6, 7, 8, 9, 10, or more times. The administrations will normally be at from one to twelve week intervals, more usually from one to four week intervals. Periodic re-administration will be desirable with recurrent exposure to the pathogen.

Currently, measurement of HAI antibody titer in serum is the most useful correlate of protection. An HAI titer of ≧40 is deemed protective, or alternatively, a four-fold increase in HAI antibody titer is considered significant (Hobson et al., 1972).

C. Antibody Production

In certain embodiments, the present invention provides H5N1 HA antigens for the production of antibodies for use in diagnostic assays. As used herein, the term “antibody” is intended to refer broadly to any immunologic binding agent such as IgG, IgM, IgA, IgD and/or IgE. Generally, IgG and/or IgM are preferred because they are the most common antibodies in the physiological situation and/or because they are most easily made in a laboratory setting.

Monoclonal antibodies (MAbs) are recognized to have certain advantages, e.g., reproducibility and/or large-scale production, and/or their use is generally preferred. The invention thus provides monoclonal antibodies of human, murine, monkey, rat, hamster, rabbit and/or even chicken origin. Due to the ease of preparation and/or ready availability of reagents, murine monoclonal antibodies will often be preferred.

However, “humanized” antibodies are also contemplated, as are chimeric antibodies from mouse, rat, and/or other species, bearing human constant and/or variable region domains, bispecific antibodies, recombinant and/or engineered antibodies and/or fragments thereof. See U.S. Pat. No. 5,482,856. The term “antibody” also is used to refer to any antibody-like molecule that has an antigen binding region, and/or includes antibody fragments such as Fab′, Fab, F(ab′)₂, single domain antibodies (DABs), Fv, scFv (single chain Fv), and/or the like. The techniques for preparing and/or using various antibody-based constructs and/or fragments are well known in the art. Means for preparing and/or characterizing antibodies are also well known in the art (see, e.g., Antibodies: A Laboratory Manual, Cold Spring Harbor Laboratory, 1988; incorporated herein by reference).

The methods for generating polyclonal and monoclonal antibodies (mAbs) generally begin along the same lines. Briefly, a polyclonal antibody is prepared by immunizing an animal with an antigen composition and collecting antisera from that immunized animal. A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies. See generally, Stills, (1994).

As is well known in the art, a given composition may vary in its immunogenicity. It is often necessary therefore to boost the host immune system, as may be achieved by coupling a peptide or polypeptide immunogen to a carrier. Exemplary or preferred carriers are keyhole limpet hemocyanin (KLH) or bovine serum albumin (BSA). Other albumins such as ovalbumin, mouse serum albumin or rabbit serum albumin can also be used as carriers. Means for conjugating a polypeptide to a carrier protein are well known in the art or include glutaraldehyde, m-maleimidobenzoyl-N-hydroxysuccinimide ester, carbodiimide or bis-biazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions. Adjuvants that may be used include IL-1, IL-2, IL-4, IL-7, IL-12, γ-interferon, GMCSP, BCG, aluminum hydroxide, MDP compounds, such as thur-MDP or nor-MDP, CGP (MTP-PE), lipid A, or monophosphoryl lipid A (MPL). RIBI, which contains three components extracted from bacteria, MPL, trehalose dimycolate (TDM) or cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants, Gerbu Adjuvant, nitrocellulose adsorbed protein, Montanide ISA, Hunter'TiterMax or aluminum hydroxide adjuvant. See, generally Bennett et al. (1992)

In addition to adjuvants, it may be desirable to coadminister biologic response modifiers (BRM), which have been shown to upregulate T cell immunity and/or downregulate suppressor cell activity. Such BRMs include, but are not limited to, Cimetidine (CIM; 1200 mg/d) (Smith/Kline, Pa.); low-dose Cyclophosphamide (CYP; 300 mg/M²) (Johnson/Mead, N.J.), cytokines such as γ-interferon, IL-2, and/or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous or intraperitoneal). The production of polyclonal antibodies may be monitored by sampling blood of the immunized animal at various points following immunization.

A second, booster injection, may also be given. The process of boosting or titering is repeated until a suitable titer is achieved. When a desired level of immunogenicity is obtained, the immunized animal can be bled and the serum isolated and/or stored, or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells or blood clots. The serum may be used as is for various applications or the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference, see also Harlow and Lane (1988). Typically, this technique involves immunizing a suitable animal with a selected immunogen composition effective to stimulate the production of antibody by B cells, followed by fusion of spleen cells with myeloma cells to form hybridomas which are then cultured or implanted into animals for the production of antibodies.

### VI. TREATMENT OF VIRAL DISEASE

Four different influenza antiviral drugs (amantadine, rimantadine, oseltamivir, and zanamivir) are approved by the U.S. Food and Drug Administration (FDA) for the treatment and prevention of influenza. All four have activity against influenza A viruses. However, sometimes influenza strains can become resistant to these drugs, and therefore the drugs may not always be effective. For example, analyses of some of the 2004 H5N1 viruses isolated from poultry and humans in Asia have shown that the viruses are resistant to two of the medications (amantadine and rimantadine). Also, please note the Jan. 14, 2006 CDC Health Alert Notice (HAN), in which CDC recommends that neither amantadine nor rimantadine be used for the treatment or prevention (prophylaxis) of influenza A in the United States for the remainder of the 2005-06 influenza season. Monitoring of avian influenza A viruses for resistance to influenza antiviral medications is ongoing.

In accordance with the present invention, a person identified as infected with influenza A may be treated with any of the aforementioned treatments, alone or in conjunction with a vaccine according to the present invention.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

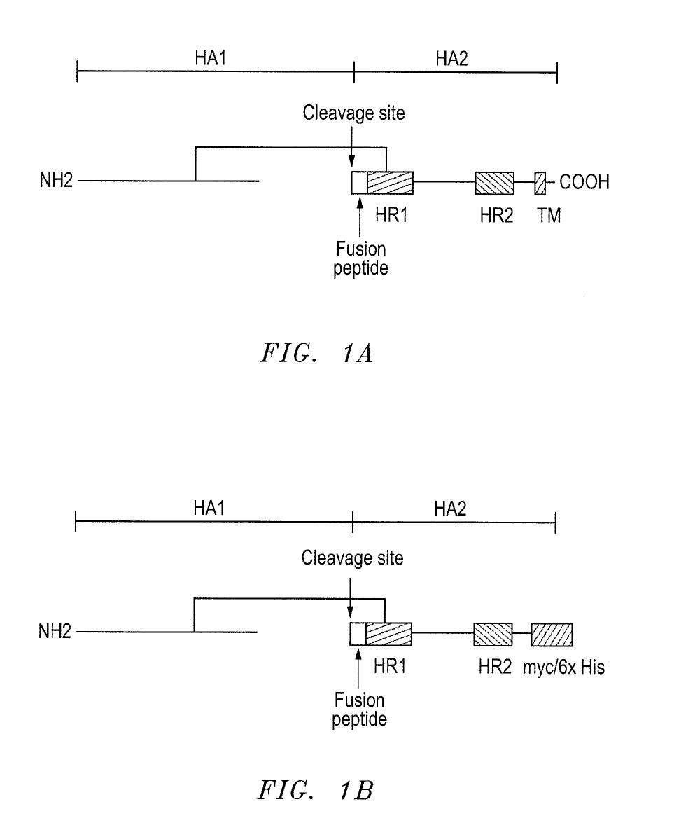

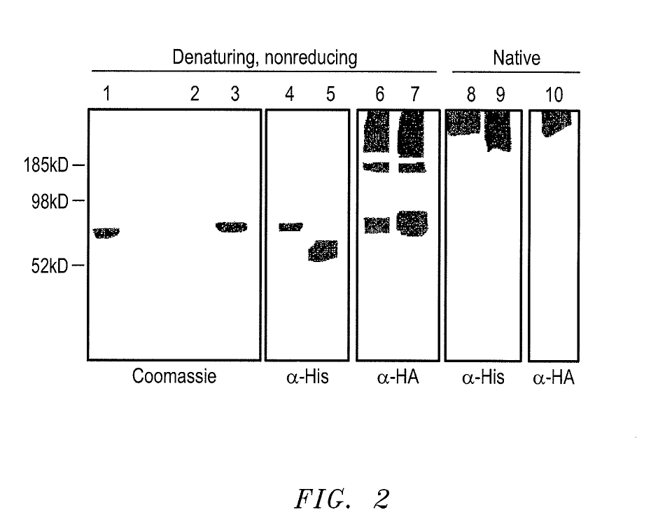

Expression of a soluble native-conformation H5 HA protein. Influenza HA proteins are synthesized as inactive precursors (HA0) that are cleaved by host-cell proteases into the biologically fusion-active HA1 and HA2 domains (FIG. 1A, next page). HA1 is extracellular and disulfide-linked to HA2. Influenza virus HA proteins are type I glycoproteins existing as trimers, with two 4-3 heptad repeat domains at the N— and C regions of the HA2 domain (HR1 and HR2), which form coiled-coil α-helices. These coiled-coils become apposed in an antiparallel fashion when the protein undergoes a low pH-induced conformational change into the fusogenic state. There is a hydrophobic fusion peptide N-proximal to the N-terminal heptad repeat, which is thought to insert into the target cell endosomal membrane, while the association of the heptad repeats brings the transmembrane domain into close proximity inducing membrane fusion (Baker et al., 1999). This mechanism has been proposed for a number of different viruses including influenza virus, RSV, and HIV (Carr and Kim, 1993; Baker et al., 1999; Lawless-Delmedico et al., 2000; Furuta et al., 1998)). HA protein is a major antigenic determinant for influenza virus, as are the analogous fusion proteins for other viruses such as HIV, RSV and Ebola virus. Thus, HA is a logical target for subunit protein vaccine strategies.

The inventors obtained sequence-optimized full-length DNA encoding the H5 influenza HA from a commercial synthesis service (GeneArt GmbH, Regensberg, Germany), based on the deduced amino acid sequence of the A/Vietnam/1203/04 strain (GENBANK accession #AY818135). The cleavage site was replaced with that of the avirulent A/teal/Hong Kong/W312/97 (H6N1). They cloned the sequence optimized H5 HA gene into a standard mammalian expression vector (pcDNA3.1, Invitrogen). This construct (DNA-HA) expresses full-length HA protein of the appropriate predicted molecular weight that is detected in a Western blot and by immunofluorescence of transfected cells by a commercially available anti-H5 HA polyclonal rabbit serum (Genetex) (data not shown). Once the expression of HA was confirmed, they designed PCR primers to amplify the extracellular portion of the H5 HA gene, deleting the transmembrane domain and cytoplasmic tail (HAΔTM, FIG. 1B). The inventors restriction cloned the HAΔTM insert into the tagged mammalian expression vector pcDNA3.1/myc-his (pcDNA/myc-his/HAΔTM), and then developed a transfection-expression system that produces highly pure milligram quantities of HAΔTM. They transiently transfected pcDNA/mychis/HAΔTM into 293 cells adapted to grow in serum-free medium in suspension (293 Freestyle, Invitrogen). The transfected cells were incubated on an orbital shaker in a CO₂ incubator. Coomassie blue staining and immunoblot analyses of transfected cell lysates and supernatant determined that peak protein expression was at 96 hours and that HAΔTM was secreted into the medium (data not shown). At 96 hours after transfection, pcDNA/mychis/HAΔTM-transfected cells were pelleted and the supernatant containing HAΔTM was purified over a HisTrap Nickel-NTA column (Amersham Biosciences) on an AKTA-design chromatography system (Amersham Biosciences). Extensive experimentation determined the optimum protocols for imidazole gradient washing, elution, fraction collection and size exclusion purification over sequential molecular weight cutoff spin concentrators (data not shown).

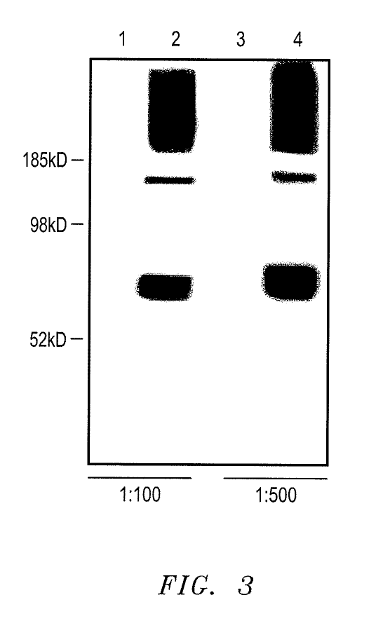

The HAΔTM construct is highly expressed in the inventors' cell culture system, with a typical yield from a 30 ml culture of 0.5-1 mg. Analysis by gel electrophoresis and protein staining demonstrates monomeric, dimeric and trimeric forms (FIG. 2, next page, lanes 1-3). The apparent molecular weight of HAΔTM is consistent with uncleaved HA0 (˜62 kD). Coomassie protein staining also shows the highly pure nature of the recombinant protein after chromatographic purification (FIG. 2, lane 3). Immunoblotting with anti-His tag antibodies (FIG. 2, lane 4) and commercially available anti-H5 HA polyclonal rabbit serum (Genetex) (FIG. 2, lane 6 and 7) also shows monomers, dimers and trimers of the predicted molecular weight. HA on virions and infected cells primarily exists in trimeric form. Analysis of the recombinant H5 HAΔTM protein under native, nondenaturing conditions show that the protein exists as a multimer, consistent with a trimer (FIG. 2, lanes 8 and 10). Trypsin digestion of HAΔTM produces two products with molecular weights consistent with HA1 (˜39 kD) and HA2 (˜24 kD), showing that HAΔTM is resistant to digestion, and suggesting that it forms a trimer. Treatment of the recombinant HAΔTM protein with peptide N-glycosidase F shows a decreased molecular weight by SDSPAGE, demonstrating that the protein is glycosylated (data not shown). These data suggest that the recombinant H5 HAΔTM protein retains important biochemical and structural characteristics of the native protein.

The commercially available rabbit anti-H5 HA serum (Genetex) was generated against a synthetic 15-amino acid peptide derived from the N-terminal sequence of the native protein. Thus, this antiserum is not directed against natively folded, intact HA protein. However, the Vanderbilt VTEU recently conducted an inactivated H5N1 vaccine trial in elderly subjects. The study vaccine was produced by chemical inactivation of egg-grown live attenuated H5N1 virus, similar to the preparation of routine seasonal influenza virus vaccines. These preparations contain native conformation HA protein and it is well established that seasonal influenza vaccines induce protective immunity. The inventors tested residual serum from an 87-year old H5N1 vaccine trial subject born in 1918 (serum kindly provided by Dr. Kathryn Edwards, Vanderbilt PI of the vaccine study) at two dilutions against H5 HAΔTM by denaturing, nonreducing SDS-PAGE and Western blot (FIG. 3). Strikingly, the post-vaccine serum detects H5 HAΔTM in monomeric, dimeric and trimeric forms. Furthermore, the post-vaccine serum detects H5 HAΔTM very strongly even at a 1:500 dilution (FIG. 3, lane 4). These data show that antibodies induced by the vaccine recognize the recombinant H5 HAΔTM and suggest that H5 HAΔTM retains elements of native conformation.

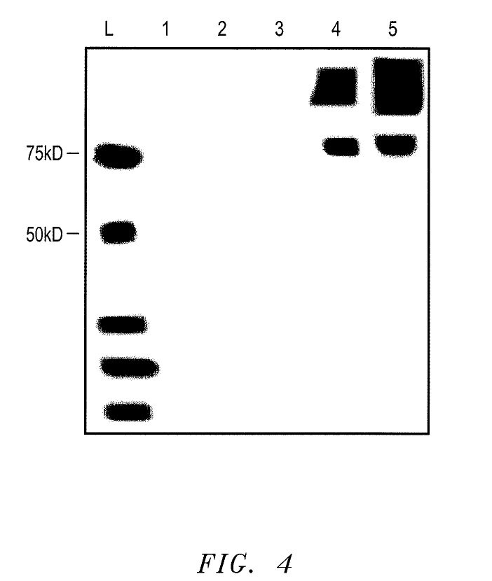

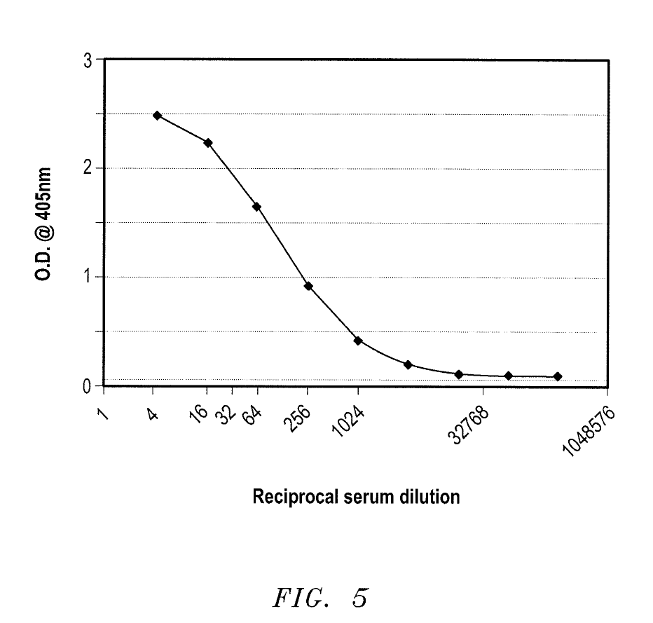

The inventors have used similar methods to express other viral glycoproteins including hMPV F (discussed below), RSV F (data not shown) and hMPV G. hMPV G is a Type II transmembrane glycoprotein, as is influenza NA, although hMPV G has not been shown to possess any enzymatic activity. Recombinant expression and purification of Type II membrane glycoproteins is often more difficult than Type I due to the transmembrane orientation and trafficking through the ER-Golgi pathway. They have successfully expressed and purified HMPV G using a similar strategy to sequence-optimize the hMPV G gene from TN/96-12, a prototype A1 strain we described previously (Williams et al., 2005). The hMPV-G construct expressed G protein of the appropriate predicted molecular weight (based on nucleotide sequence) that was detected in both immunofluorescent assays and Western blot by polyclonal anti-hMPV guinea pig serum (FIG. 4). The molecular weight in immunoblots is ˜75 kd, while the predicted mw of the primary amino acid sequence is 23 kd, suggesting hMPV-G is highly glycosylated. Thus, hMPV-specific antiserum produced against native G recognizes the recombinant hMPV-G. Native gel analysis of hMPV-G protein demonstrates that it forms multimers consistent with tetramers or larger aggregates (data not shown). This recombinant, purified protein was immunogenic and induced serum hMPV-neutralizing antibody titers in guinea pigs (data not shown). These data suggest that hMPV-G protein retains important elements of native conformation and antigenicity.

Thus, the inventors have successfully generated and purified milligram quantities of several Type I and Type II viral membrane glycoproteins proteins and characterized them by similar analytical methods including immunofluorescence, protein gel electrophoresis and immunoblot assays. All of these recombinant constructs are expressed as multimers, similar to their oligomerization state in virus-infected cells or virions.

### Example 2

Determination of serum HAI titers in human subjects. The serum antibody response to influenza virus is commonly measured using the hemagglutination inhibition (HAI) test: an HAI titer of ≧40 is deemed protective, or alternatively, a 4-fold increase in HAI antibody titer is considered significant (Hobson et al., 1972). Serum samples will be collected from HA-immunized mice to assess the serological response for comparisons with T-cell immune responses. Previously, the inventors tested subjects enrolled in a comparative influenza vaccine trial that compared trivalent inactivated influenza vaccine (TIV) with live-attenuated influenza vaccine (LAIV) for serum antibody titer to each strain of influenza virus by the HAI method, using the same antigens as those in the vaccines. The strains of influenza A/H1N1 and influenza A/H3N2 used in the two vaccines were the same, but the influenza B strains were different. Geometric mean titers (GMT) were determined in each vaccine group and were compared using the Mann-Whitney test (Table 1). The post-vaccination GMT for each of the three antigens in the 2003-2004 influenza vaccine was significantly higher in the healthy adult subjects receiving TIV when compared to the subjects receiving LAIV. Although the HAI response in LAIV subjects was lower than previous reports, these data support the observations that TIV produces significantly greater magnitude of serum HAI than LAIV (Heilman and La Montagne, 1990; Clements and Murphy, 1986; Treanor et al., 1999). These data show that we have the ability to measure serum HAI responses in H5 HA-vaccinated mice.

TABLE 1
Comparison of Serum HAI Responses in Human Subjects Receiving Either TIV or LAIV

| | A/New Caledonia (H1N1) | | A/Panama (H3N2) | | B/Hong Kong | |
|---|---|---|---|---|---|---|
| | GMT (95% CI) | | GMT (95% CI) | | GMT (95% CI) | |
| | Pre Vac | Post Vac | Pre Vac | Post Vac | Pre Vac | Post Vac |
| TIV | 26.9 (11.5-62.8) | 304 (155-597) | 71.0 (31.6-160) | 362 (195-6672) | 32.0 (17.8-57.5) | 231 (165-323) |
| LAIV | 19.0 (10.3-35.1) | 22.6 (11.9-43.1) | 48.5 (25.9-90.7) | 68.6 (39.3-120) | 24.3 (13.4-43.7) | 28.8 (17.6-47.4) |
| p-value | 0.745 | <0.0001 | 0.472 | 0.0002 | 0.515 | <0.0001 |

Preliminary development of a recombinant HAΔTM ELISA. The inventors coated Immulon II HB 96-well plates with HAΔTM at 200 ng/well in carbonate buffer. Wells were blocked with blocking buffer (PBS/Tween/5% nonfat dry milk) and then incubated with serial 4-fold dilutions of serum from an H5N1 vaccine trial subject (serum kindly provided by Dr. Kathryn Edwards, Vanderbilt PI of the vaccine study). Wells were washed and incubated with goat anti-human IgG-alk phos (Southern Biotech). After washing, PNPP substrate (Sigma) was added and O.D. was read at 405 nm on a Spectramax Pro (Molecular Devices). The vaccinee's serum exhibited strong reactivity with HAΔTM that was more than twice background up to a 1:16,000 dilution (FIG. 5).

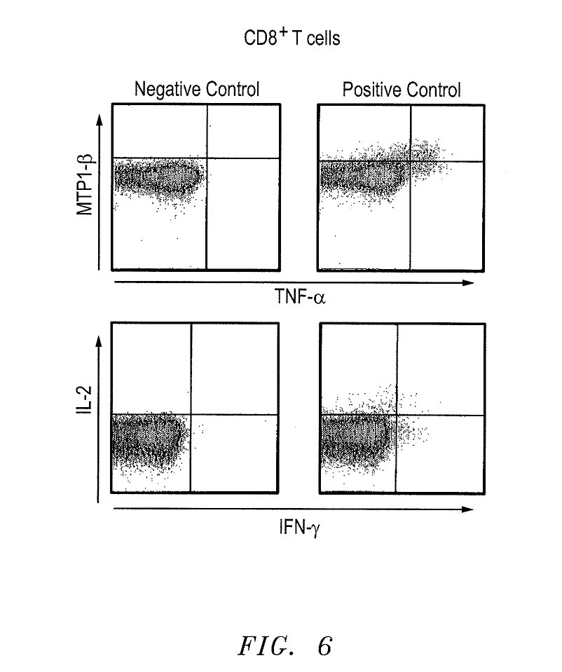

Determination of T cell responses by ELISPOT and ICS. The inventors have successfully established numerous immunological assays for evaluation of immune responses to infection and vaccination. These include intracellular cytokine staining (using 12 and more colors in a quantitative and qualitative assay, FIG. 6), ELISPOT, cytotoxicity, plaque neutralization, proliferation (CFSE, BrdU, and thymidine), cytometric bead arrays, and degranulation assays to such pathogens as influenza, RSV, pertussis, and vaccinia (Rock and Crowe Jr., 2003; Rock et al., 2005; Venter et al., 2003; Rock et al., 2004; Talbot et al., 2004; Rutigliano et al., 2005; Chen et al., 2005).

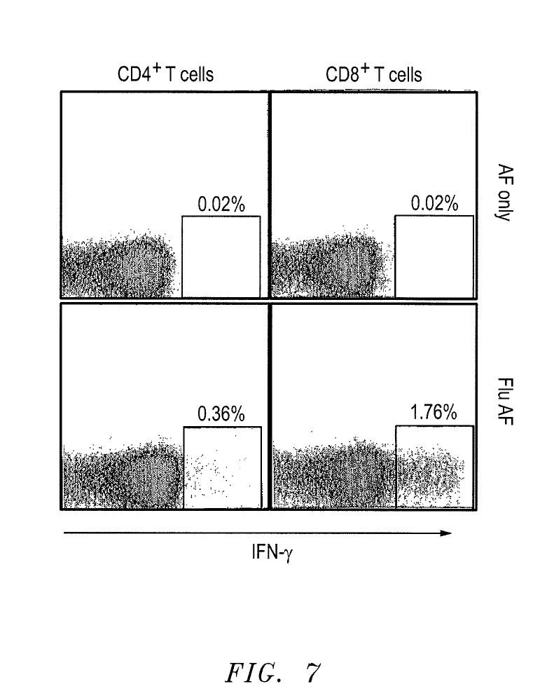

Detection of Influenza virus-specific T cells by ICS. The number of cells responding to a specific stimulus can now be determined with the advent of new functional assays. The inventors have extensive experience in developing assays and assessing T cell responses to numerous antigens and viruses (Rock and Crowe Jr., 2003; Rock et al., 2005; Venter et al., 2003; Rock et al., 2004; Talbot et al., 2004; Rutigliano et al., 2005; Chen et al., 2005) and will employ flow cytometric methods to examine the functional/phenotypic breadth of influenza virus-specific T cells in mice immunized with H5 HAΔTM. The primary strategy for assessment of influenza virus-specific T cell responses will employ a technique first described by He et al. (2003). The inventors have previously used these methods to measure the CD4+ and CD8+ T cell IFN-γ response from healthy adult subjects participating in clinical trials comparing inactivated influenza vaccine with trivalent live-attenuated vaccine. The inventors detected a positive intracellular INF-γ response against each of the three strains of influenza virus (H1N1, H3N2, and B). Representative data from these experiments using A/New Caledonia/20/99 (H1N1) is shown in FIG. 7. Overall, assessment of 20 healthy adult subjects identified an average of 0.29% influenza virus-specific T cells after A/New Caledonia/20/99 stimulation.

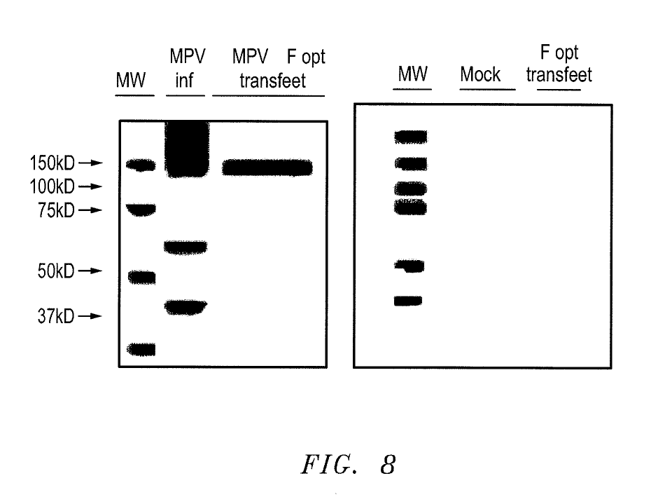

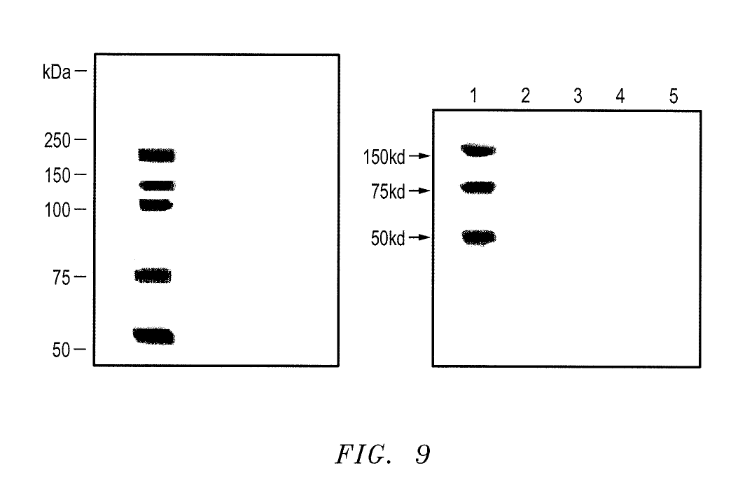

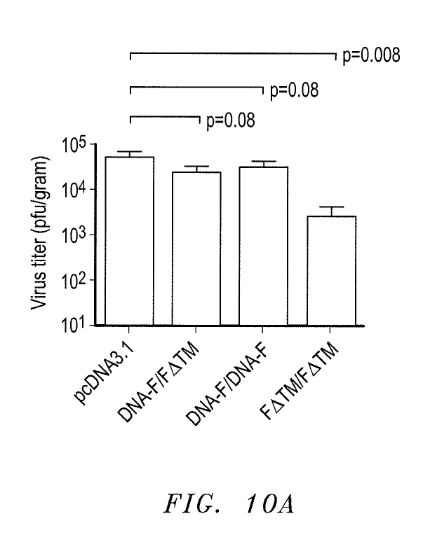

Expression of hMPV F in mammalian cells. The inventors amplified the full-length hMPV F gene from isolate TN/96-12 as described (Williams et al., 2005). The original viral sequence expressed poorly when cloned into a plasmid and transfected into mammalian cells (data not shown). The inventors then obtained optimized full-length optimized sequence from a commercial synthesis service (DNA Technologies, formerly Aptagen). They cloned the sequence-optimized hMPV F gene into a standard mammalian expression vector (pcDNA3.1, Invitrogen). This construct (DNA-F) expressed F protein of the appropriate predicted molecular weight that is detected in a Western blot by polyclonal anti-hMPV guinea pig serum that we previously generated (Williams et al., 2005) (FIG. 8). Guinea pig and human anti-hMPV serum also detect DNA-F protein by immunofluorescent assays of hMPV-infected cell monolayers (data not shown). Thus, the DNA-F construct expresses hMPV F protein of the appropriate predicted molecular weight that is detected by antiserum produced by wild-type hMPV infection, strongly suggesting that DNA-F protein possesses native conformation and glycosylation.

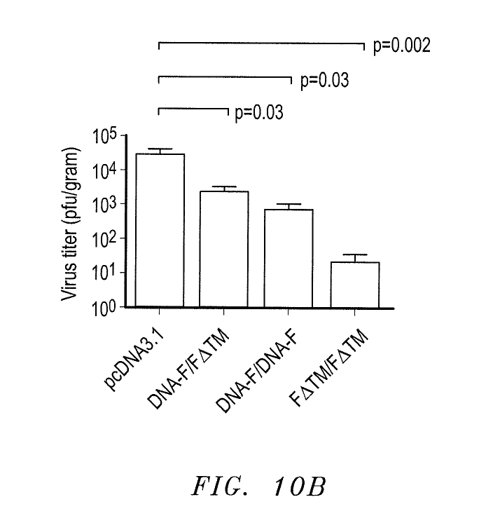

Expression of a soluble native-conformation F protein. Once the expression of Fopt was confirmed, we designed PCR primers to amplify the extracellular portion of the hMPV F gene, deleting the transmembrane domain and cytoplasmic tail (FoptΔTM). The inventors restriction cloned the FopΔTM insert into the tagged mammalian expression vector pcDNA3.1/myc-his (pcDNA/myc-his/FΔTM). The inventors utilized a transient transfection system similar to that described above for H5 influenza HA. hMPV F [symbol] TM is expressed as a trimer as analyzed by Western blot using anti-hMPV serum, and is highly pure (FIG. 9). Subsequent experiments demonstrated that the hMPV F [symbol] TM protein was highly immunogenic in cotton rats.

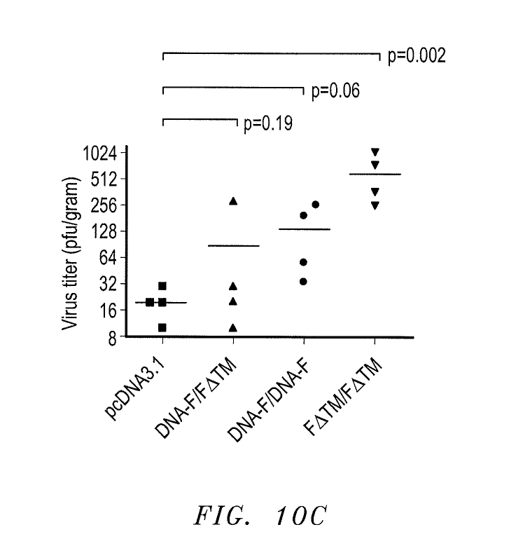

Immunization of cotton rats with DNA-Fopt or FoptΔTM and subsequent challenge with hMPV. To determine whether hMPV F was immunogenic either as a protein or as a DNA vaccine, the inventors immunized small groups of animals (4 per group) with either DNA-F alone, recombinant F protein alone, or both in a prime-boost strategy. They based the immunization schedule on studies in mice and turkeys that have shown that 2 weeks between DNA immunizations was sufficient to develop protection (Li et al., 1998; Bembridge et al., 2000; Martinez et al., 1999; Kapczynski and Sellers, 2003). These were preliminary studies designed to test the hypothesis that F alone can induce protection. The inventors did not intend in these early experiments to test protection induced by F against a variety of strains or to determine the optimum immunization regimen. They immunized cotton rats intramuscularly twice 2 weeks apart with pcDNA3.1 alone (vector control), DNA-F, or F protein (FΔTM) adjuvanted with Titermax Gold (Sigma) (Table 2). Animals were bled on day 27 for measurement of serum antibodies to hMPV. All groups except vector control group had immunofluorescent titers to hMPV-infected LLC-MK2 cells (range 1:320 to 1:1280) (data not shown). All groups were challenged subsequently intranasally with 10⁵ pfu hMPV on day 28. Four days post-infection, the animals were sacrificed and tissue titers of virus were measured by plaque assay. Animals immunized twice with FΔTM showed a modest but highly significant level of protection against shedding in nasal tissues compared to control animals (FIG. 10A), while DNAF/FΔTM and DNA-F/DNA-F groups showed reductions in nasal virus shedding that did not reach significance. Conversely, 2 doses of FΔTM protein alone were highly protective against lung virus shedding, giving a >1,500-fold reduction in mean lung hMPV titer compared to controls (FIG. 10B). DNA-F/FΔTM and DNAF/DNA-F groups showed modest but highly significant reduction of virus replication in the lungs (FIG. 10B). Cotton rats immunized with the vector control exhibited nasal and lung tissue virus replication similar to naive cotton rats during primary hMPV infection (Williams et al., 2005). The inventors measured in vitro serum neutralizing titers in all groups prior to challenge as described (Williams et al., 2005). The FΔTM/FΔTM immunized animals developed a significant rise in hMPV-neutralizing titers (mean 1:570, range 1:250-1:984, FIG. 10C). This titer was markedly higher than the mean serum neutralizing titer of 1:180 the inventors previously observed in cotton rats following primary infection with hMPV (Williams et al., 2005). The DNA-F/DNA-F group exhibited a rise in serum neutralizing titer that approached significance (FIG. 10C), while only one animal in the DNAF/FΔTM group showed a significant neutralizing antibody titer, despite this group exhibiting a significant reduction in lung virus shedding (FIG. 10B). The discrepancy between lung and nasal protection has been described in animal models of RSV (Prince et al., 1985a; Prince et al., 1985b). These results show that viral glycoproteins expressed in mammalian cells by these methods are immunogenic and can induce protective immune response in rodents.

TABLE 2
Immunization Schedule and Groups

| GROUP | DAY | IMMUNIZATION |
|---|---|---|
| Vector control | 0 | pcDNA3.1 DNA 100 μg i.m. |
| (pcDNA3.1) | +14 | pcDNA3.1 DNA 100 μg i.m. |
| hMPV F DNA/ | 0 | DNA-F DNA 100 μg i.m. |
| hMPV F DNA | +14 | DNA-F DNA 100 μg i.m. |
| hMPV F DNA prime/ | 0 | DNA-F DNA 100 μg i.m. |
| hMPV F protein boost | +14 | FΔTM 25 μg i.m. |
| hMPV F protein/ | 0 | FΔTM 25 μg i.m. |
| hMPV F protein | +14 | FΔTM 25 μg i.m. |

### Example 3

As discussed above, the inventors have generated an H5 influenza HA truncated, soluble construct (H5 HAΔTM) that is expressed at high levels in cultured mammalian cells, allowing for the production of highly pure, conformationally intact H5 influenza HA protein. The H5 HAΔTM construct is highly expressed in our system, with a typical yield from a 30 ml culture of 0.5-1 mg.

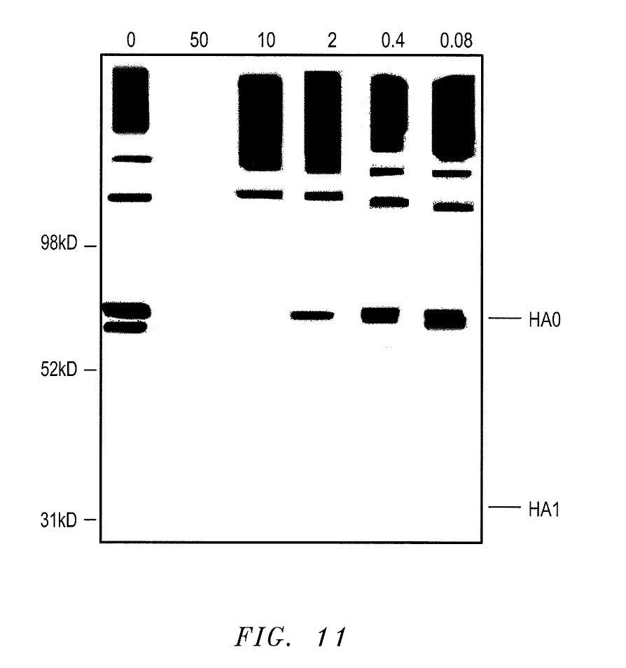

Trypsin digestion of H5 HAΔTM produces two products with molecular weights consistent with HA0 (˜62 kD) and HA1 (˜39 kD). FIG. 11 shows the effect of increasing concentrations of trypsin on the cleavability of H5 HAΔTM. At low concentrations of trypsin, only the HA0 product is detected. Increasing concentrations of trypsin cleave the HA to produce increasing amounts of HA1 (˜39 kD). HA2 [˜24 kD] is not visible on this particular blot. These results show that H5 HAΔTM is resistant to digestion, and suggesting that it forms a stable trimer.

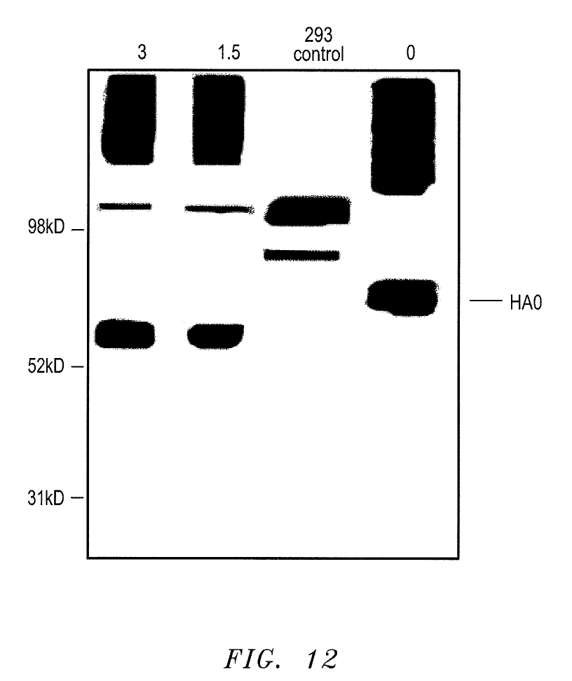

Glycosylation of the H5 HAΔTM protein was assessed. A major advantage of mammalian cell expression is the ability to generate glycosylation patterns and moieties similar to those found on native viral protein in infected human cells. This may increase the immunogenicity of H5 HAΔTM. Treatment of the recombinant HAΔTM protein with peptide N-glycosidase F shows a decreased molecular weight by SDS-PAGE (FIG. 12, lanes 1 and 2) compared to untreated protein (FIG. 12, lane 4), demonstrating that the protein is glycosylated. The discrepancy in apparent molecular weight between FIG. 12, lanes 1 and 2, suggests more complete deglycosylation with increased enzyme. Further experiments with PNG-F and Endo-H glycosidase are being performed to more fully characterize the glycosylation state of the recombinant H5 HAΔTM. Although preliminary, these data suggest that the recombinant H5 HAΔTM protein retains important biochemical and structural characteristics of the native protein.

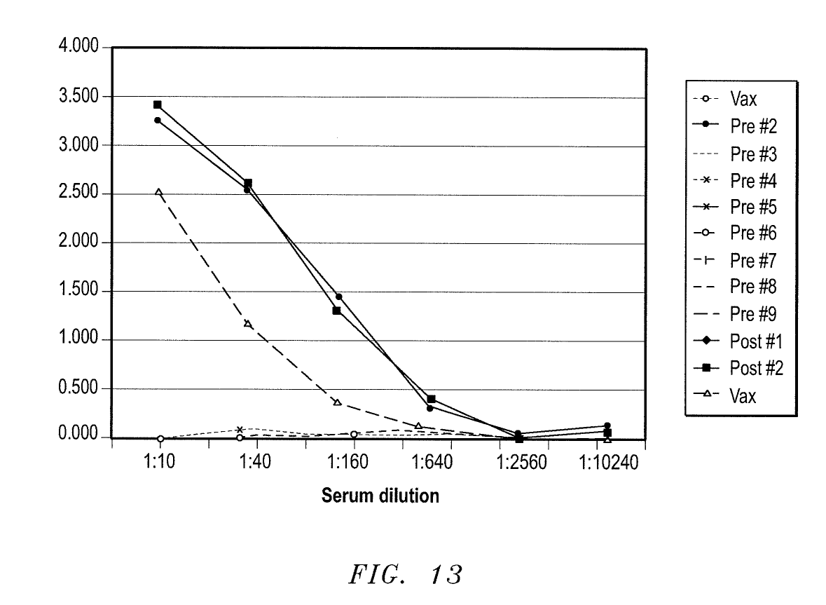

Nine 8-week-old BALB/c mice were anesthetized and injected in the quadriceps muscles with 25 μg of purified recombinant H5 HAΔTM adjuvanted 1:1 with Titermax Gold (Sigma). These initial studies were to establish immunogenicity, rather than determine optimal dose. Mice were bled at Day 0 (prior to the first dose of vaccine) and again at day 14, prior to the second dose of H5 HAΔTM. Only two animals were bled at this intermediate time point. Sera were tested for H5-specific responses by ELISA and Western blot. As shown in FIG. 13, none of the pre-immune sera showed reactivity with H5 HAΔTM by ELISA (Pre #1-9). However, the two post-immunization mice had strikingly elevated serum IgG titers to H5 HAΔTM. Although a direct comparison between mice and humans is not possible, the ELISA titer of the two mice following a single immunization of H5 HAΔTM was higher than the serum antibody titer of a subject in an H5N1 vaccine trial following two doses of vaccine (FIG. 13, Post #1-2 and Vax).

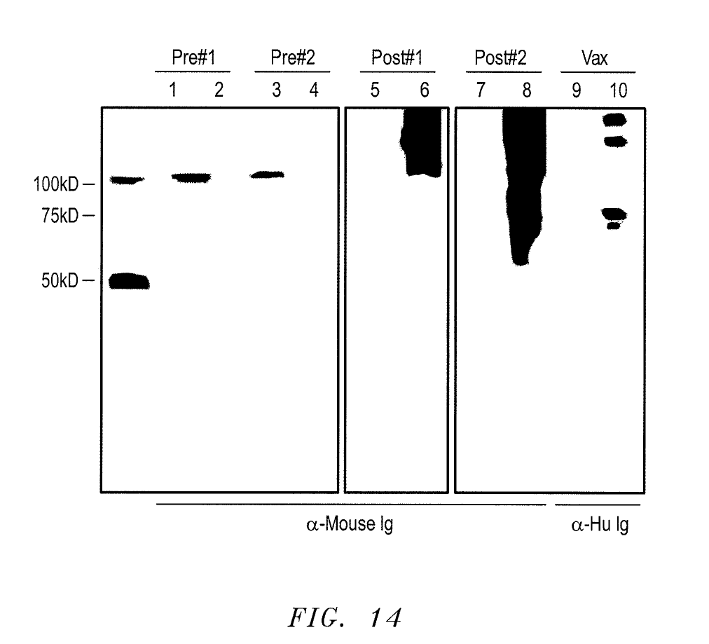

Analysis of the mouse pre- and post-immunization sera by Western blot against H5 HAΔTM showed similar results. Sera from pre-immune mice showed no reactivity to H5 HAΔTM, even at 1:100 dilution (FIG. 14, lanes 1-4). Conversely, sera from mice following one immunization detected H5 HAΔTM very strongly at 1:100 (FIG. 4, lanes 5-8). Again, although no direct comparison can be made, the mouse post-immune sera following a single immunization of H5 HAΔTM was higher than the serum antibody titer of a subject in an H5N1 vaccine trial following two doses of vaccine (FIG. 14, lanes 9-10).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 4,879,236
U.S. Pat. No. 5,482,856
U.S. Pat. No. 5,650,298
U.S. Pat. No. 5,871,986
U.S. Pat. No. 5,925,565
U.S. Pat. No. 5,935,819
Abe et al., J. Immunol., 171(3):1133-1139, 2003.
Abramson, Pediatr. Infect. Dis. J., 18(12):1103-1104, 1999.
Almendro et al., J. Immunol., 157(12):5411-5421, 1996.
Ausubel et al., In: Current Protocols in Molecular Biology, John, Wiley & Sons, Inc, New York, 1996.
Baichwal and Sugden, In: Gene Transfer, Kucherlapati (Ed.), NY, Plenum Press, 117-148, 1986.
Baker et al., Mol. Cell, 3:309-319, 1999.
Beigel et al., N. Engl. J. Med., 353(13):1374-1385, 2005.
Belshe et al., J. Infect. Dis., 181(3): 1133-1137, 2000.
Bembridge et al., J. Gen. Virol., 81:2519-2523, 2000.
Benne et al., J. Clin. Microbiol., 32:987-990, 1994.
Bennett et al., J. of Immunol. Meth., 153:31-40, 1992.
Brett et al., Virology, 339(2):273-280, 2005.
Brown et al., Semin. Immunol., 16(3):171-177, 2004.
Carbonelli et al., FEMS Microbiol. Lett., 177(1):75-82, 1999.
Carr and Kim, Cell, 73(4):823-832, 1993.
Cauthen et al., J. Virol., 74(14):6592-6599, 2000.
Chen and Okayama, Mol. Cell Biol., 7(8):2745-2752, 1987.
Chen et al., J. Virol., 79(9):5537-5547, 2005.
Clements and Murphy, J. Clin. Microbiol., 23(1):66-72, 1986.
Cocea, Biotechniques, 23(5):814-816, 1997.
Couch and Kasel, Annu. Rev. Microbiol., 37:529-549, 1983.
Couch and Kasel, J. Infect. Dis., 153:432-440, 1986.
Crawford et al., Vaccine, 17(18):2265-2274, 1999.
Crowe et al., Vaccine, 24(4):457-467, 2006.
De et al., Vaccine, 6(3):257-261, 1988.
Deml et al., J. Virol., 75:10991-1001, 2001.
Doherty et al., In: Memory and recall CD8+ T cell responses to the influenza A viruses, in Options for the Control of Influenza, Osterhaus and Cox (Eds.), Elsevier Science, NY. 293-300, 2001.
el-Madhun et al., J. Infect. Dis., 178(4):933-939, 1998.
Fechheimer et al., 1987
Fraley et al., Proc. Natl. Acad. Sci. USA, 76:3348-3352, 1979.
Furuta et al., Nat. Struct. Biol., 5:276-279, 1998.
Gabizon et al., Cancer Res., 50(19):6371-6378, 1990.
Gamblin et al., Science, 303(5665):1838-1842, 2004.
Garrison and Baker, DICP, 25(6):617-627, 1991.
Ghendon et al., Vaccine, 23(38):4678-4684, 2005.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Glaser et al., J. Virol., 79(17):11533-11536, 2005.
Gopal, Mol. Cell Biol., 5:1188-1190, 1985.
Gossen et al., Proc. Natl. Acad. Sci. USA, 89:5547-5551, 1992.
Gossen et al., Science, 268(5218):1766-1769, 1995.
Graham and Van Der Eb, Virology, 52:456-467, 1973.
Ha et al., EMBO J., 21(5):865-875, 2002.
Ha et al., Virology, 309(2):209-218, 2003.
Halvorson, Avian Pathol., 31(1):5-12, 2002.
Harland and Weintraub, J. Cell Biol., 101(3):1094-1099, 1985.
Harlow and Lane, In: Antibodies: A Laboratory Manual, Cold Spring Harbor Laboratory, Cold Spring Harbor, N.Y., 346-348, 1988.
Hatta et al., Science, 293(5536): 1840-1842, 2001.
He et al., J. Infect. Dis., 187(7):1075-1084, 2003.
Heilman and La Montagne, Pediatr. Clin. North Am., 37(3):669-688, 1990.
Hobson et al., J. Hyg (Lond), 70(4):767-777, 1972.
Horimoto et al., J. Virol., 77(14):8031-8038, 2003.
Horimoto et al., Virology, 213(1):223-230, 1995.
Kaneda et al., Science, 243:375-378, 1989.
Kapczynski et al., Avian Dis., 47:1376-1383, 2003.
Karaca et al., Clin. Diagn. Lab. Immunol., 12(11):1340-1342, 2005.
Kraus et al. FEBS Lett., 428(3):165-170, 1998.
Kuroda et al., EMBO J., 5(6):1359-1365, 1986.
Kuroda et al., Virology, 174(2):418-429, 1990.
Kurodaetal., Virology, 180(1):159-165, 1991.
Lakey et al., J. Infect. Dis., 174(4):838-841, 1996.
Lareyre et al., J. Biol. Chem., 274(12):8282-8290, 1999.
Lawless-Delmedico et al., Biochemistry, 39:11684-11695, 2000.
Lee et al., Biochem. Biophys. Res. Commun., 238(2):462-467, 1997.
Levenson et al., Hum. Gene Ther., 9(8):1233-1236, 1998.
Li et al., J. Exp. Med., 188:681-688, 1998.
Liu et al., Virology, 314(2):580-590, 2003.
Lu et al., J. Virol., 79(11):6763-6771, 2005.
Macejak and Samow, Nature, 353:90-94, 1991.
Mackenzie et al., Immunology, 67(3):375-381, 1989
Maniatis, et al., Molecular Cloning, A Laboratory Manual, Cold Spring Harbor Press, Cold Spring Harbor, N.Y., 1990.
Martinez et al., Eur. J. Immunol., 29:3390-3400, 1999.
McMichael et al., J. Gen. Virol., 67(Pt 4):719-726, 1986.
McMichael et al., N. Engl. J. Med., 309(1):13-7, 1983.
Neuzil et al., N. Engl. J. Med., 342(4):225-231, 2000.
Nguyen et al., J. Virol., 74(12):5495-5501, 2000.
Nguyen et al., Virology, 254(1):50-60, 1999.
Nichol, Vaccine, 19(31):4373-4377, 2001.
Nicolas and Rubinstein, In: Vectors. A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nomoto et al., Gene, 236(2):259-271, 1999.
Okuno et al., J. Clin. Microbiol., 28:1308-1313, 1990.
Olsen et al., Vaccine, 15(10):1149-1156, 1997.
Oxford et al., Vaccine, 23(46-47):5440-5449, 2005.
Palker et al., Virus Res., 105(2):183-194, 2004.
Pelletier and Sonenberg, Nature, 334(6180):320-325, 1988.
Potter et al., Proc. Natl. Acad. Sci. USA, 81:7161-7165, 1984.
Powers et al., J. Infect. Dis., 175(2):342-351, 1997.
Prince et al., J. Virol., 55:517-520, 1985b.
Prince et al., Virus Res., 3:193-206, 1985a.
Quinlivan et al., J. Virol., 79(13):8431-8439, 2005.
Remington U.S. Pat. No. 5,980,912
Ridgeway, In: Vectors: A Survey of Molecular Cloning Vectors and Their Uses, Rodriguez et al. (Eds.), Stoneham: Butterworth, 467-492, 1988.
Rippe, et al., Mol. Cell Biol., 10:689-695, 1990.
Rock and Crowe, Jr., et al., Immunology, 108(4):474-480, 2003.
Rock et al., J. Immunol., 174(6):3757-3764, 2005.
Rock et al., J. Immunology, 174(6):3757-3764, 2005.
Rock et al., J. Infect. Dis., 189(8):1401-1410, 2004.
Rock et al., J. Infect. Dis., 189(8):1401-1410, 2004.
Rutigliano et al., Virology, 337(2):335-343, 2005.
Sambrook et al., In: Molecular cloning, Cold Spring Harbor Laboratory Press, Cold Spring Harbor, N.Y., 2001.
Stech et al., Nat. Med., 11(6):683-689, 2005.
Stephenson and Zambon, Occup. Med. (Lond), 52(5):241-247, 2002.
Stills, In: The Biology of the Laboratory Rabbit, Manning et al. (Eds.), Academic Press, Inc. NY, 435-448, 1994.
Subbarao et al., Virology, 305(1):192-200, 2003.
Swain et al., Viral Immunol., 17(2):197-209, 2004.
Swayne et al., Vaccine, 18(11-12):1088-1095, 2000.
Talbot et al., JAMA, 292(10):1205-1212, 2004.
Talbot et al., JAMA, 292:1205-1212, 2004.
Taubenberger et al., Nature, 437(7060):889-893, 2005.
Taubenberger et al., Philos. Trans. R Soc. Lond. B Biol. Sci., 356(1416):1829-1839, 2001.
Taylor and Askonas, Immunology, 58(3):417-420, 1986.
Taylor et al., Vaccine, 6(6):504-508, 1988.
Temin, In: Gene Transfer, Kucherlapati (Ed.), NY, Plenum Press, 149-188, 1986.
Treanor et al., J. Infect. Dis., 173(6):1467-1470, 1996.
Treanor et al., Vaccine, 18(9-10):899-906, 1999.
Treanor et al., Vaccine, 19(13-14):1732-1737, 2001.
Tsumaki et al., J. Biol. Chem., 273(36):22861-22864, 1998.
Tumpey et al., Avian Dis., 48(1):167-176, 2004.
Tumpey et al., Science, 310(5745):77-80, 2005.
Tur-Kaspa et al., Mol. Cell Biol., 6:716-718, 1986.
Vanlandschoot et al., Arch. Virol., 141(9):1715-1726, 1996.
Venter et al., J. Virol., 77(13):7319-7329, 2003.
Wagner et al., Glycobiology, 6(2):165-175, 1996.
Wagner et al., J. Virol., 70(6):4103-4109, 1996.
Wang et al., Vaccine, 24(12):2176-2185, 2005.
Wareing and Tannock, Vaccine, 19(25-26):3320-3330, 2001.
Webster et al., Virology, 149(2):165-173, 1986.
Williams et al., J. Virology, 79:10944-10951, 2005.
Wu and Wu, Biochemistry, 27: 887-892, 1988.
Wu and Wu, J. Biol. Chem., 262:4429-4432, 1987.
Wu et al., Biochem. Biophys. Res. Commun., 233(l):221-226, 1997.
Yang and Russell, Proc. Natl. Acad. Sci. USA, 87:4144-4148, 1990.
Zambon, Rev. Med Virol., 11(4):227-241, 2001.
Zhao-Emonet et al., Biochim. Biophys. Acta, 1442(2-3):109-119, 1998.

1. A nucleic acid sequence comprising SEQ ID NO:1 (full length) or SEQ ID NO:3 (extracellular).

2. The nucleic acid sequence of claim 1, further comprising a promoter active in eukaryotic cells.

3. The nucleic acid sequence of claim 2, wherein said promoter is a constitutive, tissue specific or inducible promoter.

4. The nucleic acid sequence of claim 1, further comprising a nucleic acid sequence encoding a vector.

5. The nucleic acid sequence of claim 4, wherein the vector is a viral vector.

6. The nucleic acid sequence of claim 5, wherein the viral vector is a retroviral vector, an adenoviral vector, an adeno-associated viral vector, a herpesviral vector, a pox-viral vector or an influenza viral vector.

7. The nucleic acid sequence of claim 4, wherein the replicable vector is a non-viral vector.

8. The nucleic acid sequence of claim 4, wherein said nucleic acid sequence is embedded in a lipid vehicle.

9-10. (canceled)

11. A method of expressing a protein in a cell comprising:
(a) providing a mammalian host cell comprising a nucleic acid sequence comprising SEQ ID NO:1 or SEQ ID NO:3, wherein said nucleic acid sequence is under the control of a promoter active in said mammalian host cell; and
(b) culturing said mammalian host cell under conditions supporting protein expression.

12. The method of claim 11, further comprising isolating said protein.

13. The method of claim 12, wherein isolating comprises one or more of solubilizing said cell, chromatography, and/or treatment with enzymes that degrade non-proteinaceous molecules.

14. The method of claim 11, wherein providing comprising transforming a mammalian host cell with a vector comprising SEQ ID NO:1 or SEQ ID NO:3 and said promoter.

15. The method of claim 14, wherein said vector is a viral vector.

16. The method of claim 14, wherein said vector is a non-viral vector.

17. The method of claim 14, wherein said mammalian host cell expresses said protein transiently.

18. The method of claim 14, wherein said nucleic acid sequence integrates into the genome of said mammalian host cell.

19. The method of claim 12, further comprising admixing the purified protein with an adjuvant.

20. The method of claim 12, further comprising lyophilizing the purified protein.

21. An isolated recombinant influenza HA oligomeric protein retaining native influenza H5 HA structure.

22. (canceled)

23. A vaccine comprising a recombinant influenza HA oligomeric protein retaining native influenza HA structure and an adjuvant in a pharmaceutically acceptable buffer.

24-25. (canceled)

26. A method for inducing an immune response in a subject comprising contacting said subject a recombinant influenza HA oligomeric protein or fragment thereof retaining native influenza H5 HA structure.

27. The method of claim 26, wherein contacting comprises administering said recombinant influenza HA oligomeric protein or fragment thereof to said subject.

28. The method of claim 26, wherein contacting comprises administering a vector encoding said recombinant influenza HA oligomeric protein or fragment thereof to said subject.

29. The method of claim 26, wherein said subject is a human.

30. The method of claim 26, further comprising measuring a humoral and/or cellular immune response is said subject after contacting.
